(19) United States
(12) Patent Application Publication
(10) Pub. No.: US 2026/0241030 A1
(43) Pub. Date: Aug. 20, 2026

(54) MULTIFUNCTIONAL HYBRID CELLULOSE NANOCRYSTALS-GOLD NANOPLATFORM AND USE THEREOF IN MEDICINE

(71) Applicants: UNIVERSITÀ DEGLI STUDI DI FIRENZE, Firenze (IT); ISTITUTO EUROPEO DI ONCOLOGIA S.R.L., Milano (IT)

(72) Inventors: Saverio MINUCCI, Opera (MI) (IT); Cristina GARIBALDI, Peschiera Borromeo (MI) (IT); Riccardo CAZZOLI, Latina (LT) (IT); Amal Kamal Said ABDELAZIZ SAADELDIN, Heliopolis (EG); Roberto ORECCHIA, Pino Torinese (TO) (IT); Barbara RICHICHI, Sesto Fiorentino (FI) (IT); Giacomo BIAGIOTTI, Campi Bisenzio (FI) (IT); Debora BERTI, Sesto Fiorentino (FI) (IT); Patrizia ANDREOZZI, Firenze (IT); Sergio Enrique MOYA, San Sebastián (ES)

(21) Appl. No.: 18/869,698

(22) PCT Filed: May 30, 2023
(86) PCT No.: PCT/IB2023/055515

§ 371 Date: Nov. 26, 2024

(30) Foreign Application Priority Data
May 31, 2022 (IT) 102022000011450

(51) Int. Cl. A61K 47/69; A61K 33/242; A61P 35/00

(52) U.S. Cl. CPC A61K 47/6939; A61K 33/242; A61P 35/00

(57) ABSTRACT

The present invention refers to a multifunctional CNC*-Au-based nanoplatform, related conjugates and/or composition containing thereof.

The present invention also refers to the use of such a multifunctional CNC*-Au-based nanoplatform, related conjugates and/or composition containing thereof in medicine

### STATE OF THE ART

Cellulose nanocrystal (CNC) is a low-cost functional and smart material, which can be easily prepared by controlled acid hydrolysis from different renewable sources.

In particular, cellulose nanocrystals (CNC) are the derivative of native cellulose which can be obtained through acid hydrolysis of the native cellulose, where the cellulose is exposed to sulfuric acid under controlled temperature and time period.

According to an easy representation, the native cellulose molecules derive from the fibrils of natural plant cells coming from trees and nanocrystals of cellulose are then obtained through acid hydrolysis from there.

Owing to its peculiar biocompatibility, biodegradability and good chemical stability the cellulose nanocrystal (CNC) has emerged as one of the most appealing natural biopolymer to such an extent that it has great potential to be considered as the ‘magic bullet’ for medicine, especially nanomedicine.

Small spindle-like size, stiff-chain architecture and large surface area (250-500 m²/g), are unique features that allowed the preparation of CNC-based advanced nanomaterials for a plethora of application areas spanning across material and life science.

With regard to biological systems, the large surface area and the surface hydroxyl groups enable easy functionalization and bioconjugation with specific biomolecules which allow targeting disease sites. Then, the known protein-repellent properties of carbohydrate-based polymers make CNC suitable to serve as nanocarriers for the development of therapeutics, resulting in a prolonged circulation in the bloodstream and delayed opsonization by phagocytic cells.

In addition, it should be noted that the rod-shaped morphology and surface chemistry significantly affect CNC fate: CNC exhibits slower rate of renal clearance and, interestingly, for drug delivery applications the untargeted nanocrystals showed a low uptake in several cell types. Then, the stiff-chain architecture makes CNC an excellent platform for the fabrication of metal nanohybrids. Of note, from a general perspective, it is known that material composition, physicochemical properties and therefore surface chemistry can significantly affect the interactions with immune cells in terms of nanomaterial fate and at some extent of induction or tolerance.

Among them, CNC-gold composites (CNC-Au) are an attractive example of hybrid nanomaterials where the complementary and synergic combination of the physiochemical properties of gold nanoparticles (AuNPs) and CNCs resulted in unique materials with catalytic and sensing performances.

Three main methodologies for hybrid CNC-Au composites have been reported so far and they include: (i) in situ fabrication of AuNPs in a matrix of CNC, (ii) mixing of preformed AuNPs with CNC, (iii) seeded growth protocol where the AuNPs are enlarged on step-by-step basis. Then, less common protocols such as, for example, solid grinding, micro-patterning, inkjet-printing, electrospinning, e-beam evaporation, dip coating and layer-by-layer assembly have been recently proposed for specific applications.

In particular, the in situ fabrication of AuNPs includes the adsorption of the gold salt (i.e. tetrachloroauric acid, HAuCl₄) onto CNC matrix, followed by a reduction of the salt, mainly using sodium borohydride (NaBH₄), to metallic gold. However, to ensure a narrow and homogeneous AuNPs distribution, the use of chemically modified CNC with molecules that works as reductant or stabilizer is commonly applied. Therefore, a pre-treatment of the cellulose nanocrystals (CNCs) by anchoring such modifying molecules to the OH groups of the glucose residues of the CNC is required. Thus, this pre-treatment step results on a significant structural modification of the initial CNC composition and surface chemistry such as, for example, CNC quaternarization, PEGylating, acetylation, derivatization with polyamidoamine (PAMAM) dendrimers that impart positive charges on CNC surface, or with poly(2-(dimethylamino) ethyl methacrylate) (PDMAEMA) chains, or poly(diallyldimethylammoniumchloride) (PDDA), or radical-polymerization reactions on CNC surface. The mentioned modifications of the CNC surface lead to the drawback of significantly modifying its physical characteristics and properties, thus impacting the CNC functions that could be obtained and the nano-biointeractions.

The Chinese patent CN111803629A discloses a nano-cellulose crystal-based organic-inorganic hybrid multifunctional biological material characterized in that it is mainly composed of nano-cellulose crystals (CNC) derivatized using polydopamine (PDA) coating. The CNC is entirely covered by the polydopamine, thus concealing the cellulose stick and consequently preventing the interaction with the cells that are able to see the polydopamine only. According to the Chinese patent the cellulose nanocrystals (CNC) are used as a template, and a layer of polydopamine is wrapped on the surface of the CNC forming a film. The FIG. 1 of CN111803629A shows the CNC covered by a dark colored layer that is represented by the polydopamine. In addition to that, it is known in the art that although the polydopamine is biocompatible it is also characterized by its own defined properties that are quite different from those of the cellulose. Polydopamine is an adhesive polymer, able to absorb light. It is quite different from the cellulose and has its own functionalities, thus masking the cellulose.

In the Chinese patent the cellulose covered by the polydopamine is furthermore added with long chain polyethylene glycol (PEG—5000 Da). In other words, the cellulose of the Chinese patent does not interact at all with the cells.

The biocompatibility of any cells with the cellulose is modified, because the relationship becomes with the polydopamine or with any other modification agent covering the cellulose stick and not with the cellulose itself. The original characteristics of the cellulose are masked, somehow lost due to the presence of the modifications. This Chinese patent discloses how the OH groups of the polydopamine are able to coordinate the gold nanostars (AuNSs). CN111803629A refers to the use of the cellulose nanocrystals as a base material, and polydopamine as coupling material which is used as a bridge between nanocellulose crystals and nano gold stars. In order to achieve the result the Chinese patent discloses a multi-step process, leading to a significant level of complexity of the manufacturing process and modifications of the biocompatibility.

All these modifications significantly impact the composition and the properties of the pristine CNC affecting also the possibility to further functionalize the OH groups of the glucose residues of the CNC with specific biomolecules which allow targeting disease sites. The resulting CNC-Au composites do not include additional anchoring points for further functionalization of the PEGylated CNC-Au composite. The lack of possibility to further functionalize the PEGylated CNC-Au composite is intended as a significant side effect of the composite.

Examples of modified celluloses used to prepare hybrid CNC-Au composites can be represented by cellulose ether, cellulose lipoate, thiolabeled cellulose, quaternary ammonium, cellulose triacetate as well as cellulose-polidopamine. Cellulose-dopamine is, in fact, the substance disclosed in the above mentioned CN111803629A. Notably, often the preparation of the CNC-Au composites requires multistep protocols that include high reaction temperatures (i.e. hydrothermal reaction at 120° C. for 10 h) or the use of ionic liquid, and these are considered as undesired effects.

Some of the above modifications of the CNC surface are also not compatible with the next synthetic steps required to stabilize the CNC suspensions, so affecting also the stability and reproducibility of the CNC-Au composites, leading to the need of having in place controlled reaction conditions (i.e for cellulose lipoate). This is another side-effect of complex processes and materials.

It is therefore known in the art that modified celluloses loose the original biological and physical characteristics, affecting the biocompatibility, the stability, the reproducibility and many other features of the cellulose material such the CNCs and CNCs-based compounds for which the natural characteristics are instead considered something to be preserved for get a good results in the following uses.

On the other hand, small sized and stable AuNPs can be prepared by using the established Brust-Schiffrin-like methodology that consists on the reduction of tetrachloroauric acid (HAuCl₄) with sodium borohydride (NaBH₄) in presence of a thiol-ending ligand that ensures the formation of an Au—S bond. In some cases, the lipoic acid is used to stabilize the gold particles but its use is currently known only in positions preventing the following functionalization of the cellulose material.

There are articles reporting studies on the sulfation of cellulose α-lipoate and the ability of the sulfated groups to stabilize colloidal suspensions of the resulting CNC-Au composites, but the reaction conditions of the sulfatation protocol are not compatible with the functionalities included in the cellulose lipoate. Indeed, the lipoic acid itself is oxidated in an undesired mode.

It is well known in the art as the complexity of the material and of the process for obtaining the material are undesired and unfavorable to get the goal of having something ready to use, able to maximize the effect especially on a medicine and nanomedicine approach where there is a significant need to obtain the results in an easy, quick and reliable way. In addition, complexity of the process/material can impart the batch-to-batch reproducibility of the manufacturing process and thus the transferability of the results in the clinical applications.

If the tumor treatments, especially solid tumor treatments, are considered as target point it is clear how the possibility to transfer the results in the clinical applications is very important; it is known in the art that for having a product ready for the market reproducibility and high definition are requested.

In this connection, radiotherapy is one of the most employed technique for the treatment of solid tumors. Radiotherapy uses high-energy radiation to induce cellular damage, slowing tumor growth by damaging their genetic material.

However, tumor radio-resistance (mainly due to tumor-associated hypoxia) and the sparing of surrounding healthy tissues remain a challenge. Many efforts have been focused in the past on the improvement of the therapeutic ratio of radiotherapy in terms of targeted radiation delivery and specific tumor sensitization.

Radiosensitizers are considered magic bullets as they increase the radiation dose deposition in the tumor site thereby allowing to use lower and safer radiation doses to non-target tissues for a same effect on tumors. In particular, gold nanoparticles (AuNPs) are excellent radiation absorbers, thanks to their higher energy absorption coefficients compared to soft tissues. When interacting with radiation, they mainly enhance electrons release and production of reactive oxygen species. The enhancement of radiotoxicity on tumor cells which have internalized the AuNPs has been demonstrated either in vitro and in vivo.

Several approaches for the targeted delivery of the AuNPs at the tumor site have been undertaken in the last years to deal with improving NPs biodistribution, ensuring an increased therapeutic window.

Gold nanoparticles (AuNPs) size, shape and surface coating strongly affect cell uptake, biodistribution, and blood circulation shelf-life. For this reason, small sized (preferably ≤5 nm) AuNPs are promising radiation dose enhancers, although the low driving force for their uptake and their rapid renal clearance limit translation into clinics. Larger AuNPs (20-60 nm) showed a maximum cell uptake whereas they accumulate in the reticulo endothelial system leading to undesirable side effects. The inherent limitations are also related to AuNPs' colloidal stability and biodistribution which are strongly affected either by medium conditions (i.e., pH, ionic strength) and interactions of NPs surface with biomolecules, which induce NPs aggregation and wrapping (i.e., formation of protein corona shell on NPs surface). There is therefore the need to find an improved material, without reduced disadvantages.

The present inventors were able to surprisingly found an easy, affordable, stable, modular and improved multifunctional hybrid CNC-Au-based nanoplatform.

The present inventors also surprisingly found a scalable straightforward one-step process that obtain, in a unique way, such an improved multifunctional CNC-Au-based nanoplatform.

It should be noted that the term “cellulose nanocrystal (CNC)” according to the invention refers to pristine natural cellulose nanocrystals as starting material, preferably acquired on the market as CelluForce NCC™ (here-below also CNC**), while the term cellulose nanocrystals after controlled sulfation as disclosed below refers to cellulose nanocrystals prepared within this invention starting from CelluForce NCC™ (here-below also CNC*).

### Definitions

CNC Cellulose nanocrystals obtained from different renewable and waste source CNC* Sulfatated cellulose nanocrystals prepared starting from the commercially available CelluForce NCC™ (here-below also CNC**).

CNC** Commercially available CelluForce NCC™.

CNC-Au Hybrid nanomaterial that consists of gold nanoparticles embedded into cellulose nanocrystals matrix (both CNC* and CNC**).
NPs nanoparticles
AuNPs gold nanoparticles
LA lipoamide

CNC-Au-LA Hybrid nanomaterial that consists of gold nanoparticles embedded into cellulose nanocrystals matrix (both CNC* and CNC**) and bearing a lipoamide spacer linked on gold surface through the formation of an Au—S bond and provided with a terminal alkyne moiety.

CNC-Au-Tm Hybrid nanomaterial that consists of gold nanoparticles embedded into cellulose nanocrystals matrix (both CNC* and CNC**) and bearing a lipoamide spacer linked on gold surface through the formation of an Au—S bond.

The lipoamide spacer is linked to a tumor targeting moiety (Tm) through a tetrazole group.

Tm Tumor targeting moiety. In some examples of this invention Tm are glucose (Glc) or mannose (Man) or lactose (Lac) residues bearing a PEG spacer at the anomeric position (in case of lactose at the terminal monosaccharide (Glc) residue).

The Tm is linked to the lipoamide spacer on CNC-Au-LA surface through a tetrazole group.

HEPES 4-(2-hydroxyethyl)-1-piperazineethanesulfonic acid, it is a buffer used in cell culture to better stabilize and maintain physiological pH conditions.

Dulbecco's Modified Eagle Medium (DMEM) is a common used basal medium to grow many mammalian cells in vitro.
DEF Dose enhancement factor
SER sensitive enhancement ratio
BODIPY-like fluorescent probe is a derivative of the 4,4′-difluoro-4-bora-3a,4a-diaza-s-indacene

CNC*-Au-BODIPY Hybrid nanomaterial that consists of gold nanoparticles embedded into cellulose nanocrystals matrix prepared from CNC** and bearing a lipoamide spacer linked on gold surface through the formation of an Au—S bond.

The lipoamide spacer is linked to a BODIPY-like fluorescent probe through a tetrazole group.

CNC*-Au-Glc-BODIPY Bifunctional hybrid nanomaterial that consists of gold nanoparticles embedded into celluloce nanocrystals matrix prepared from CNC** and bearing a lipoamide spacer linked on gold surface through the formation of an Au—S bond. The lipoamide spacer is linked to a glucose residue (Glc) and to a BODIPY-like fluorescent probe through a tetrazole group.

### BRIEF DESCRIPTION OF THE FIGURES

FIG. 1 shows the schematic representation of the CNC*-Au-LA composite described in this invention and the versatility of the proposed nanomaterials in terms of multiple sites of functionalization, with bioactive headgroups and functional stabilizers, of both the OH groups of the CNC and of the alkyne residues on the AuNPs surface.

FIG. 2 shows the synthetic strategy employed to prepare the CNC*-Au-LA described in this invention.

FIG. 3 shows Transmission Electron Microscopy of the CNC*-Au-LA. AuNPs size was evaluated according with Schneider, C. A., Rasband, W. S., Eliceiri, K. W. “NIH Image to ImageJ: 25 years of image analysis”. Nature Methods 9, 671-675, 2012. AuNPs 5±nm.

FIG. 4 shows the chemistry that can be employed for the conjugation of bioactive headgroups and functional stabilizers on the CNC*-Au-LA.

FIG. 5 shows the synthetic strategy employed to prepare the monofunctional and the bifunctional CNC*-Au conjugates with the bioactive headgroups by means of iterative step-by-step conjugation reactions described in this invention.

FIG. 6. shows the schematic representation of the CNC*-Au conjugates described in this invention. In particular, Tm bearing composites where Tm=Glucose (i.e., CNC*-Au-Glc) or Tm=Mannose (i.e., CNC*-Au-Man) or Tm=Lactose (i.e., CNC*-Au-Lac); bioactive headgroups ═BODIPY (as representative example of fluorescent probe, i.e., CNC*-Au-BODIPY) or bioactive headgroups=2-Aminoethanesulfonic acid, (named Taurine) (as representative example of the possibility to further functionalize the CNC*-Au-LA on the OH groups of the CNC*, i.e., CNC*-Au-Taurine); bifunctional conjugate bearing two bioactive headgroups (i.e., CNC*-Au-Glc-BODIPY, and CNC*-Au-LA-Taurine-BODIPY). Reaction conditions: a. CuSO₄:sodium ascorbate, H₂O, Azido-PEG-Tm (where Tm=Man, or Glc) (2.0 eq); b. CuSO₄:sodium ascorbate, H₂O, Azido-PEG-Glc (0.3 eq); c. CuSO₄:sodium ascorbate, DMF, BODIPY (0.5 eq); d. CuSO₄:sodium ascorbate, H₂O, Azido-PEG-Lac (1.5 eq); e. CuSO₄:sodium ascorbate, H₂O, Azido-PEG-Lac (0.5 eq); f CuSO₄:sodium ascorbate, H₂O, Azido-PEG-Lac (0.15 eq); g. CuSO₄:sodium ascorbate, DMF, BODIPY (0.1 eq); h. DMSO, carbonyldiimidazole (CDI), Na₂CO₃.

FIG. 7 shows CNC*-Au (left) and CNC**-Au (right) resuspended as described in material and methods, concentration 1.0 mg/ml in water, pictures have been acquired on EVOS fl microscope (Thermofisher) without filters. In left panel magnification of 40× has been reached without observing major signs of aggregation (white bar represents 100 μm), in right panel magnification of 10× (white bar represents 400 μm).

FIG. 8 shows the schematic representation of the CNC**-Au conjugates described in this invention. In particular, Tm bearing composites where Tm=Glucose (i.e., CNC**-Au-Glc) or Tm=Mannose (i.e., CNC**-Au-Man) or Tm=Fucose (i.e., CNC**-Au-Fuc). Reaction conditions: a. CuSO₄:sodium ascorbate, H₂O, Azido-PEG-Tm (where Tm=Man, or Glc, or Fuc) (2.0 eq).

FIG. 9. CNC**-Au-Glc (left) and CNC**-Au-Man (right) resuspended as described in material and methods, concentration 10 ug/ml in complete cell growth medium (DMEM+NA (North American fetal bovine serum)) after 24 h, pictures have been acquired on EVOS fl microscope (Thermofisher) without filters. Magnification of 4× has been reached observing major signs of aggregation (white bar represents 1000 μm).

FIG. 10 shows image of atomic force microscopy (AFM) image of the sulphated CNC*.

FIG. 11 shows AuNPs uptaken by human triple negative breast cancer cells (MDA-MB-231 cell line) and healthy breast cells (MCF-10A cell line). Cells were treated with CNC*-Au-LA, CNC*-Au-Glc and CNC*-Au-Man.

FIG. 12 shows surviving fraction in log 10 of MDA-MB231 (upper panels) and MCF10a (lower panels) colonies, after combinatory treatment of x-ray and CNC*-Au functionalized with either mannose (left panels) or glucose (right panels) at described concentrations as reported in the Figure legends. Data from 2 independent experiments with 2 replicates each (n=4).

FIG. 13 Turbidimetry assay on CNC*-Au-Man 0.05% with ConA 10 μM in 10 mM HEPES (pH 7.4), 1 mM MnCl₂; B. Turbidimetry assay on CNC*-Au-Man 0.05% with ConA at various concentrations in 10 mM HEPES (pH 7.4), 1 mM MnCl₂ after 60 minutes.

FIG. 14 Normalized luminescence of MDA-MB231 (left panel) and MCF10a (right panel) cells treated with increasing concentrations of CNC*-Au-Glc and CNC*-Au-Man (0, 2.5, 5, 10, 20, 50 and 100 μg/ml) for 24 hrs without Xrays, measured by cell titer glo as described in material and methods. 3 replicates each concentration (n=3).

Table 1 (FIG. 17) shows four tables divided by cell type (MDA-MB231 upper tables and MCF10a lower tables) and by CNC*-Au-LA functionalization with mannose (left tables) or glucose (right tables). Columns represent concentrations of CNC*-Au conjugates treatments, DEF, SER and uptake value μg/liter calculated on 3 replicates and relative standard deviation.

FIG. 15 shows surviving fraction in log 10 of A375 melanoma cells colonies, after combinatory treatment of x-ray and CNC*-Au-Man and CNC*-Au-Glc functionalized with either mannose (left panels) or glucose (right panels) at described concentrations as reported in the Figure legends. Data from 2 independent experiments with 2 replicates each (n=4).

FIG. 16 Normalized luminescence of A375 melanoma cells treated with increasing concentrations of CNC*-Au-Glc and CNC*-Au-Man (0, 2.5, 5, 10, 20, 50 and 100 μg/ml) for 24 hrs without Xrays, measured by cell titer glo as described in material and methods. 3 replicates each concentration (n=3).

Table 2 (FIG. 18) shows two tables for A375 melanoma cells divided by CNC*-Au-LA functionalization with mannose (left table) or glucose (right table). Columns represent concentrations of CNC*-Au-Glc and CNC*-Au-Man treatments, DEF, SER and uptake value μg/liter calculated on 3 replicas and relative standard deviation.

FIG. 19 shows flow cytometric analysis of U251 glioblastoma cells viability by propidium iodide exclusion assay 24 hours after combinatory treatment of x-ray and CNC*-Au-Glc at 20 μg/mL (graph A) and surviving fraction in log 10 by colony-forming assay of U251 glioblastoma cells after combinatory treatment of x-ray and CNC*-Au-Glc at 10 μg/mL (graph B).

### DESCRIPTION OF THE INVENTION

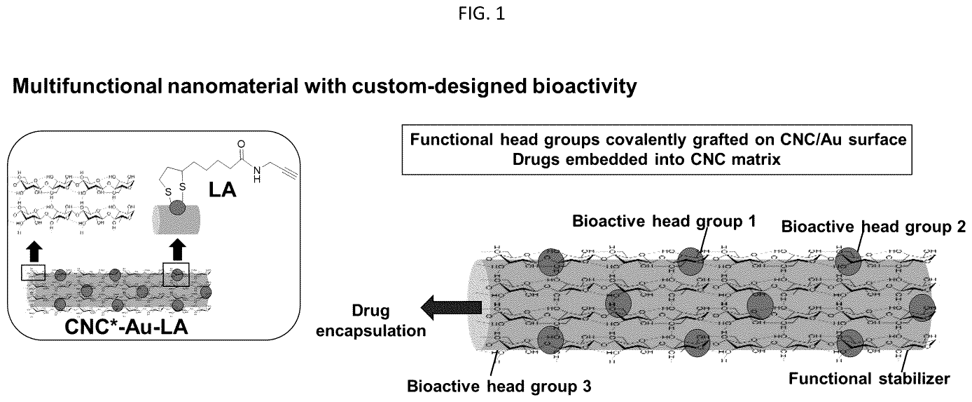

The present invention refers to easy, affordable, stable, modular and improved multifunctional CNC*-Au-based nanoplatform, related conjugates and/or composition containing thereof (FIG. 1).

The present invention also refers to the use of such a multifunctional CNC*-Au-based nanoplatform, related conjugates and/or composition containing thereof in medicine, preferably nanomedicine. More preferably, the present invention refers to the use of such a multifunctional CNC*-Au-based nanoplatform, related conjugates and/or composition containing thereof in the treatment of tumors, preferably solid tumors and more preferably solid tumors selected among the group consisting of breast cancers, uterine cancers, brain and central nervous system cancers, lung cancers, liver cancers, kidney cancers, testicular cancers, pancreatic cancers, skin cancers, thyroid cancers, intestinal cancers, head and neck cancers, ovarian cancers, stomach cancers, colon cancers, bladder cancers, prostate cancers, and urinary tract cancers, eye cancers, oral and oropharyngeal cancers, HIV/AIDS-related cancers, hematopoietic and lymphoid cancers, and sarcomas of various tissues.

Much more preferably, breast cancer is represent by triple negative breast cancer, brain and central nervous system cancers is represented by glioblastoma and skin cancer is represented by melanoma.

Furthermore, the present invention also refers to the use of such a multifunctional CNC*-Au-based nanoplatform, related conjugates and/or composition containing thereof in radiotherapy, immunotherapy, photodynamic therapy, boron neutron capture therapy (BNCT) and in cancer diagnosis.

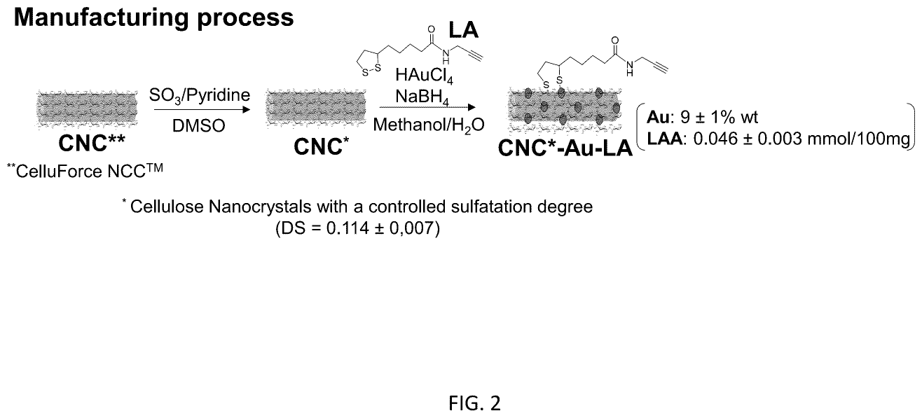

The present invention further refers to a scalable straightforward one-step process that obtain the multifunctional CNC*-Au-based nanoplatform with improved characteristics (FIG. 2).

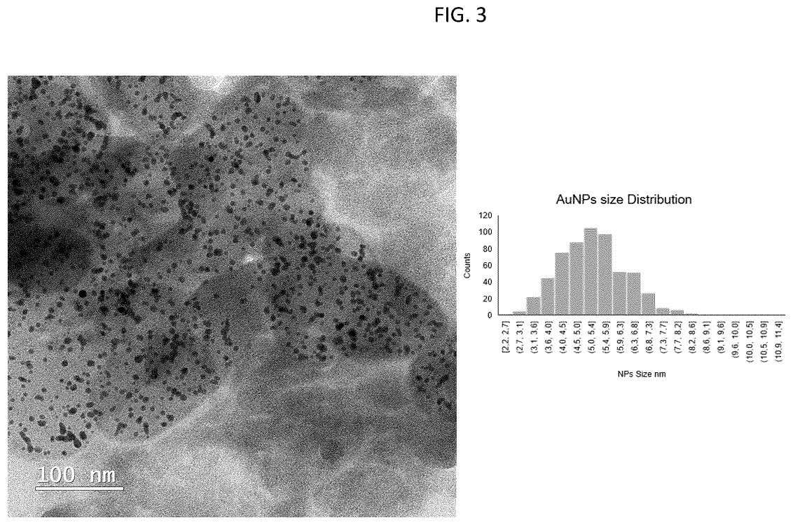

According to the present invention, small gold-nanoparticles (AuNPs) bearing a spacer with a terminal chemical reporter are stably embedded into a cellulose nanocrystal (CNC*) matrix (FIG. 1-2). The dimensions of the gold-nanoparticles according to the invention are preferably ≤5 nm (FIG. 3).

According to the present invention, a spacer is preferably a bifunctional monodentate or bidentale thioalkyl-bearing molecule such as mercaptoacetic acid (MAA), mercaptosuccinic acid (MSA), mercaptopropionic acid (MPA) as monodentante molecules, whereas as bidentate the α-lipoic acid (DHLA) or its derivative i.e. a α-lipoic acid conjugated to the primary amine group of an alkyne amine (i.e lipoamide (LA) in this invention) or of an ethylenediamine-N,N-diacetic acid residue (EDADA).

Lipoamide (LA) according to the invention is a monocarboxylic acid amide resulting from the formal condensation of the carboxy group of the α-lipoic acid with propargyl ammine (FIG. 1-2). Terminal chemical reporter according to the invention are alkyne residues, such as for example propyne, butyne, cyclooctine and its derivatives such as OCT, MOFO, DIFO, DIMAC, COMBO, DIBO, DIBAC, BARAC, BCN, TMTH.

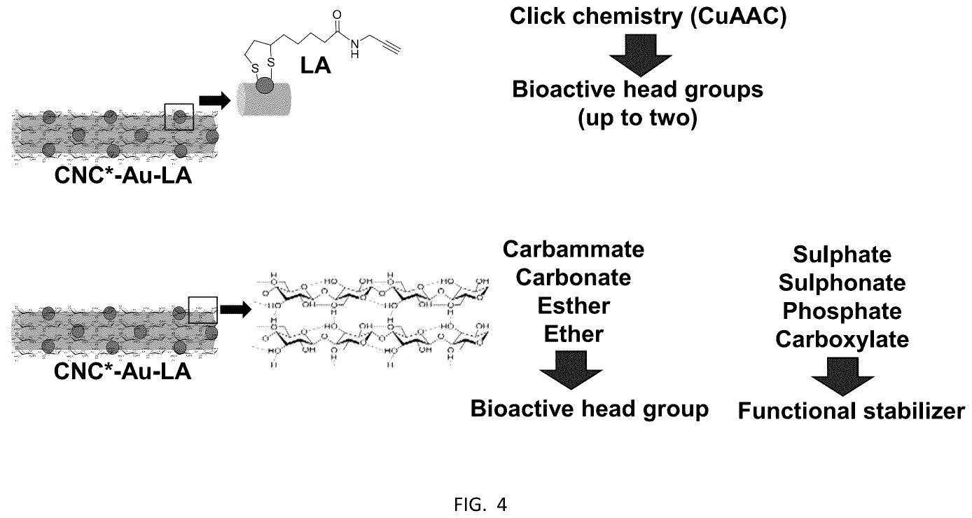

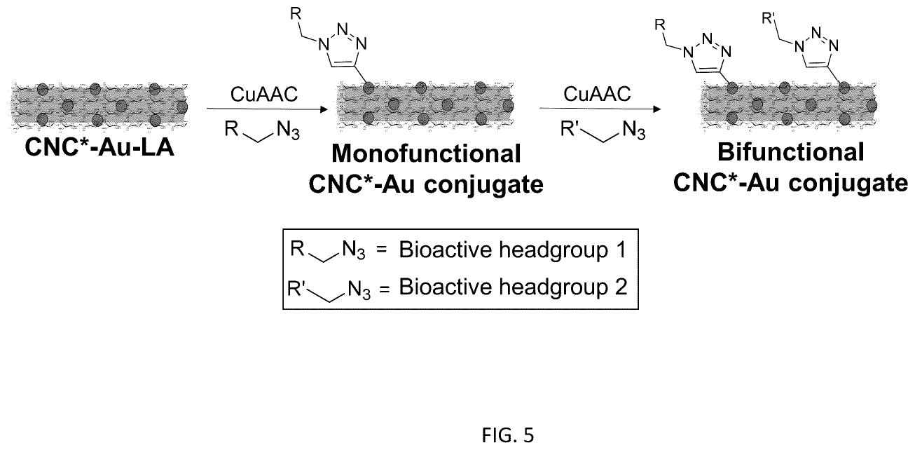

It results in a hybrid smart nanoplatform of cellulose nanocrystals gold-nanoparticles, named CNC*-Au-LA (FIG. 1-2). The hybrid cellulose nanocrystal-gold nanoparticles of the invention are construed to offer at least one and up to two easily accessible and orthogonal anchoring points on its surface, that are preferably represented by the OH groups of the glucose residues of the CNC and the alkyne on Au surface (FIG. 5). The term “hybrid” refers to gold nanoparticles (AuNPs) and CNCs together. Accordingly this invention provides a CNC-Au composite where the surface modifications do not significantly impact the composition and the properties of the pristine CNCs whereas ensures the presence of anchoring point for further multiple functionalization of the CNC surface (FIG. 4).

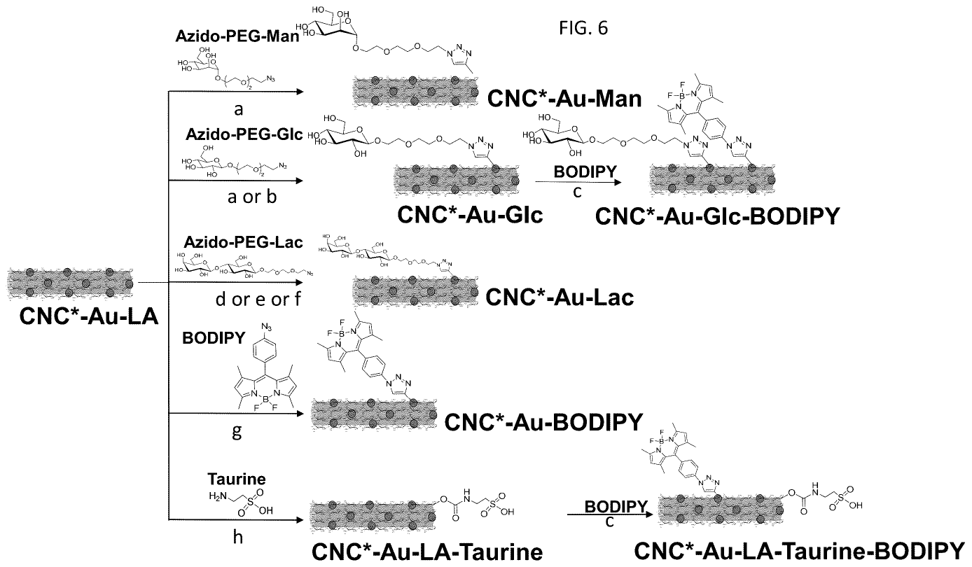

Indeed, according to the invention, these anchoring points can be harnessed for the covalent conjugation of at least one and preferably up to three structurally different functionalities. According to a preferred embodiment of the invention, said at least one and up to three functionality is represented by at least one bioactive headgroup together with an additional functional stabilizer (CNC*-Au conjugates in FIG. 6 where the functional stabilizer are sulfate groups).

According to the invention, the hybrid smart nanoplatform of cellulose nanocrystals gold-nanoparticles (CNC*-Au-LA) with the at least one up to three functionalities is considered as a cellulose nanocrystal gold-nanoparticles material containing targeting molecule/s (CNC*-Au-Tm, FIG. 6). Targeting molecules (Tm) as below are discussed in terms of bioactive headgroups. Then, BODIPY fluorescent probe (FIG. 6) and has been selected as model example of the possibility to conjugate up to two bioactive headgroups on gold surface. Then, the 2-aminoethanesulfonic acid (Taurine) has been selected as model example of the possibility to conjugate a bioactive headgroups on CNC surface despite the presence of the functional stabilizer (FIG. 6).

The bioactive headgroups (as targeting group) and the gold-nanoparticles (AuNPs) according to the invention define the biological identity of the hybrid nanomaterial providing cellulose nanocrystal conjugates able to expert specific, ad-hoc biological properties also with the ability to reach desired sites of action; thanks to their characteristics these conjugates can be employed in the treatment of different diseases giving an improved efficacy decreasing the off-target side-effects, especially giving the effect of delivering the nanomaterial to the specific desired site of action, preferably to the tumor site for a specific tumor treatment.

According to the present invention, said bioactive headgroup can include structurally different bioactive molecules that define the biological activity of the material and can be selected among the group consisting of diseases associated i) antigens such as, for example, tumors, pathogens including bacteria and virus and/or autoimmune diseases; ii) immune cells targeting molecules such as, for example, saccharides (including mono-, di- tri-, tetra- and oligosaccharides), nucleotides, lipids, peptides; iii) targeted receptors such as, for example, carbohydrate-binding receptors (i.e. DC-SIGN, Langherin, Mincle, Mannose receptor); peptide-binding receptors; nucleotide-binding receptors (i.e. Toll-like receptors) and/or lipid-binding receptors (i.e. CD1d, Toll-like receptors); iv) molecules that target tumor associated receptors such as, for example the glucose transporter proteins (GLUTs), mannose receptor, folate receptor, CD44, galectin, ASGP receptors (see example of CNC*-Au-Man, CNC*-Au-Glc, CNC*-Au-Lac, FIG. 6); v) fluorescent probes (see example CNC*-Au-BODIPY, FIG. 6); vi) radionuclide probes for PET and/or SPECT; and vii) boron cage molecules.

According to the invention, preferred bioactive headgroups are carbohydrate, more preferably sugars and even more preferably selected between glucose and mannose, lactose, fucose, rhamnose, Lewis-type antigens (i.e. Lewis^{a}, sialylLewis^{a}, Lewis^{X}, sialylLewis^{X}, Lewis^{b}, sialylLewis^{b}), glucuronic acid, glycomimetics. According to a preferred embodiment, the functionalization are three functionalizations and encompasses glucose (Glu), mannose (Man) and lactose (Lac).

According to the present invention, the design and the preparation of the CNC*-Au-LA has been defined taking into account parameters that could ensure the bench-to-bedside perspective, meaning the transfer from the basic research to the application of the research itself. In particular: a) the colloidal stability of the nanoparticles dispersions in different media; b) the manufacturing process in terms of (i) costs, (ii) green and scalable protocols with a high batch-to-batch reproducibility (FIG. 2); c) the fine control of the loading (see example of CNC*-Au-Lac, CNC*-Glc-BODIPY, FIG. 6) of the functional molecules on NPs surface are a crucial bottle neck that, in general, significantly affects the bench-to-bedside translation of the NPs.

In addition, the increasing complexity of the multifunctional NPs is something that need to be carefully taken into account when attempting the grafting of multiple cargos on nanoparticles (NPs) surface. Indeed, time and cost consuming step-by-step protocols that include multiple conjugation reactions and purifications procedures often result in low yields, difficulty on scale-up and potential heterogeneities in formulation.

According to the invention, it was also surprisingly found a manufacturing process to obtain the hybrid nanomaterial able to comply with all the functionalities allowing the presence of the bioactive headgroup/s (see FIG. 1 and FIG. 5-6).

At first, the manufacturing process of the invention provides that the gold nanoparticles (AuNPs) have been prepared in situ, according to a one-pot process (as shown in FIG. 2). The single step, called one-pot, is very surprising and represent a big advantage avoiding undue complexities.

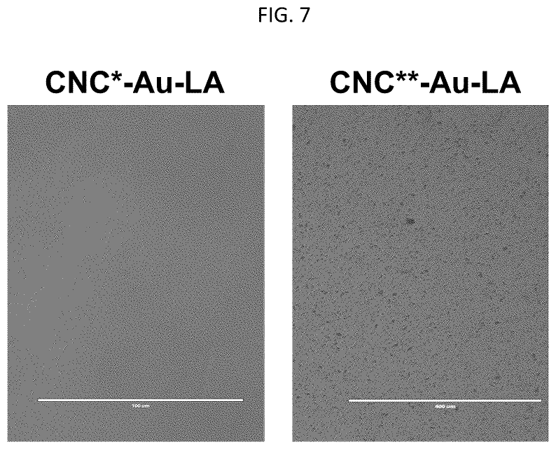

The manufacturing process according to the present invention comprises: i) using cellulose nanocrystals (CNC) as starting material; the starting material is very important to guarantee a final improved nanoplatform (CNC* vs CNC**, FIG. 7); ii) using a spacer (i.e. LA) as gold stabilizer and sodium borohydride (NaBH₄) to reduce gold salts as tetrachloroauric acid (HAuCl₄). According to a preferred embodiment, the spacer of the invention can be a monodentate spacer or bidentate spacer, where monodentate means that there is one single sulphide group forming the Au—S bond having terminal groups such as carboxy, amine and azides and bidentate means that there are two sulphide groups such as for example the lipoic acid or the DHLA-EDADA; more preferably the spacer of the invention is the α-lipoamide (LA).

The spacer is installed on the gold surface in situ in a one-pot process with the aid of a solvent, being preferably a solution containing methanol/water, in a ratio from 20:1 to 10:1, preferably a ratio of about 10:1 (FIG. 2). The solvent was crucial for installing the spacer on the gold (Au) surface in situ in the one-pot process according to the invention; this ensures the dispersibility of all the reaction components (FIG. 2). The spacer according to the invention is the α-lipoamide (LA) and the methanol:water mixture is in a ratio of 10:1.

In more detail, the process of the invention comprises:
a) Preparation of CNC*: treating the pristine cellulose nanocrystals with a sulfatation agent that is preferably a mixture of SO₃/pyridine and a solvent that is preferably the dimethylsulfoxide (DMSO) to give cellulose nanocrystals with a controlled sulfation (FIG. 2);
b) Preparation of CNC*-Au-LA: treating the CNC* with a controlled sulfation as obtained at step a) with the lipoamide (LA), the HAuCl₄/NaBH₄ in a solution of methanol/water to give the hydryd nanoplatform of CNC*-Au-LA (FIG. 2).

According to the invention the preparation of CNC*-Au-LA of step b) as above is performed as a one pot reaction.

The resulting CNC-Au-LA according to the process of the invention is then easily purified by dialysis as final passage.

According to this process the small particle size distributed AuNPs, with dimensions preferably ≤5 nm, more preferably of about 5.0±1.0 nm (FIG. 3), are stably embedded onto the cellulose nanocrystals (CNCs) matrix, avoiding the need of additional derivatization steps on the CNCs and ensuring the one-pot step.

The possibility to avoid additional derivatizations is a great improvement, simplifying the process and maximizing the efficiency of the obtained nanoplatform. The skilled man, in fact, considers any additional derivatization step that is usually necessary to obtain homogeneous nanoparticles distributions as an undesired complexity, known as affecting the composition and the properties of the final nanoplatform.

For instance, the above mentioned Chinese patent CN111803629A discloses the use of the polydopamine (PDA) for additional derivatization of the cellulose material, having the OH groups of the polydopamine acting to coordinate the gold particles, leading to a favorable AuNPs growing. This as said above leads to a layer of polydopamine that is wrapped on the surface of the CNC with a significant profile of complexity. No other possibility of functionalization is disclosed in CN111803629A.

According to the invention the gold-nanoparticles (AuNPs) surface is provided with the terminal reactive group for the custom engineering of the cellulose nanocrystal-gold-α-lipoamide material surface (CNC*-Au-LA surface, FIG. 2 and FIG. 5-6), that ensures the versatility of the nanoplatform and the fine tuning of its biological activity.

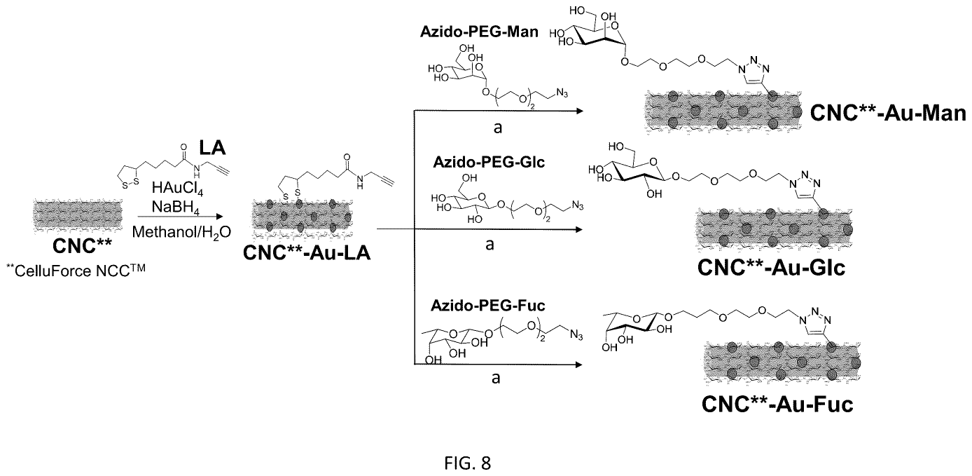

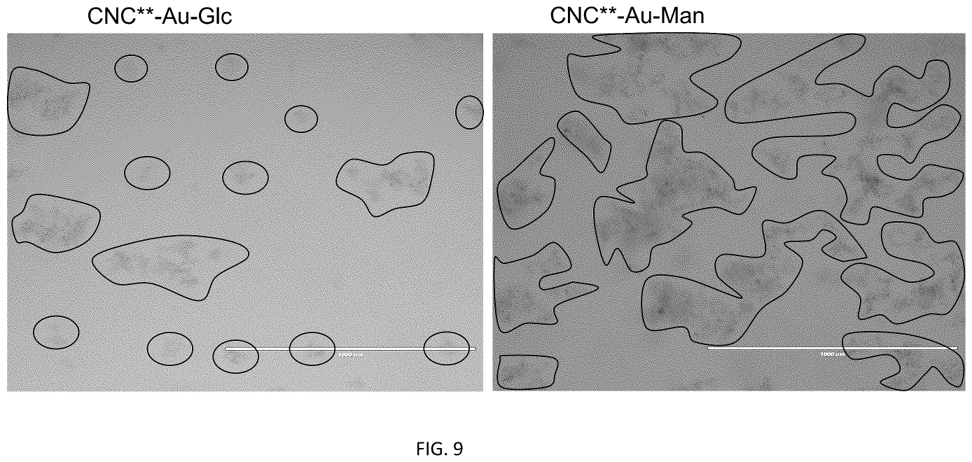

For the cellulose nanocrystal (CNC), different acids can be employed for their preparation and its selection may affect the dispersity and colloidal stability of cellulose nanomaterials (CNCs). Indeed, surface charge (g) affects the colloidal stability of the nanoparticles (NPs) and in particular, the cellulose nanomaterials derived from sulfuric acid hydrolysis disperse readily in water. In a very first attempt for the preparation of the CNC-Au-LA the inventors used directly a pristine CNC** (CelluForce NCC™). It was a sulfated cellulose nanomaterial in which there is roughly one sulfate group each 6 glucose residues, with a g-potential of −39 mV±2 and hydrodynamic diameter of 70 nm. However, at that time it was noted that the reaction conditions used for accessing CNC-Au-LA affected the dispersibility of the resulting hybrid CNC**-Au-LA nanomaterial owing to a reduction of some sulfate groups in these conditions; this effect corresponds to a drawback described in the literature on similar CNC substrates and it moved the inventors to modify the structure of the CNC** to improve dispersibility. It resulted in the formation of aggregates of the CNC**-Au-LA water dispersions, that were confirmed by dynamic light scattering analyses (hydrodynamic diameter of CNC**-Au-LA=2935±462 nm vs pristine CNC**=70 nm) causing low reproducibility in the further functionalization steps and significantly limited the biological applications of the NPs. Then, any attempt to improve the dispersibility of the CNC**-Au-LA and the stability in water failed and significant aggregation of the material occurred (FIG. 7). However, the functionalization of the CNC**-Au-LA was performed according to the invention through an azide-alkyne cycloaddition reaction (FIG. 8), eventually in presence of a catalyst that can be represented by copper. Preferably, in case copper is used as a catalyst of the reaction is in form of CuSO₄. In particular, Tm such as glucose (Glc), Mannose (Man), and Fucose (Fuc) glycosides bearing a PEG spacer at the anomeric position were used for the conjugation. However, the significant aggregation of the CNC**-Au conjugates was observed in complete cell growth media (FIG. 9). Therefore, such composition of the CNC**-Au composite did not allow to further assess the biological activity of the conjugates with cells.

Therefore, a careful design of the composition of the CNC used as starting material was required to overcome this issue. In order to preserve the structure and composition of the CNC avoiding modifications that may affect the general unique properties of this nanomaterial, the inventors surprisingly found that by modulating the sulfation degree of the pristine CNC** it was possible to have minimal modifications, using the same functional groups already included in the pristine CNC**.

The sufatation degree (DS) imparted water dispersibility and consequently colloidal stability of the CNC dispersions in water. However, high sulfation degree (DS) resulted in higher negatively charged NPs surface (lower g-potential values) that may also affect biological properties such as toxicity and cell uptake, and macromolecular conformation that can directly influence the physicochemical features. The fine tuning of the sulfation degree of CNC was therefore a further challenging step. The present inventors were surprisingly able to manage the sulfation of the cellulose nanocrystals obtaining the homogeneity of the reaction mixture, avoiding any safety issue due to the use of complex reagents such as, for example, the chlorosulfuric acid.

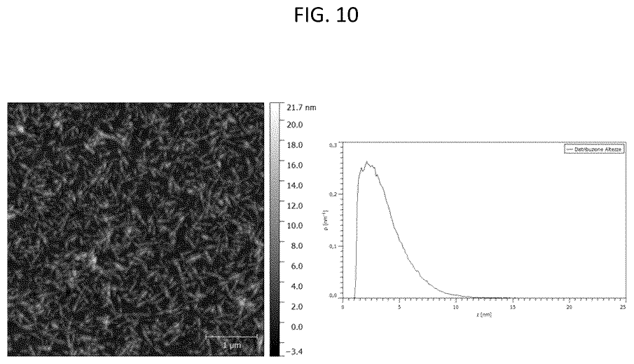

In addition, the inventors were able to control the standard deviation (DS) of the sulfation degree of the cellulose nanoparticles and the undesired and severe depolymerization of the cellulose nanoparticles. The CNC* (FIG. 10) was analyzed by atomic force microscopy (AFM) and it showed that CNC* maintains the same shape of the pristine CNC** in these conditions.

All the parameters related to the type of pristine cellulose nanocrystals (CNCs) employed as starting materials of the invention are relevant to obtain the desired nanoplatform; in particular, the preferred molecular weight is between 14700 Da and 27850 Da and the initial source is preferably pristine cellulose (CelluForce NCC™.

According to a preferred embodiment of the present invention, the cellulose nanocrystal obtained after sulfatation (CNC* as reported in FIG. 2) have a sulfatation degree (DS) of 0.114±0.007 (DS of the pristine cellulose−CelluForce NCC™ (CNC**)=0.028). The sulfation of the invention is scalable and it provides cellulose nanocrystals with controlled sulfation (CNC*) bearing additional sulfate groups as functional stabilizer, with high batch-to-batch reproducibility. The sulfatation step according to the present invention is performed on the cellulose nanocrystals using a commercially available and safe sulfation reagent, preferably represented by SO₃-pyridine or SO₃—NEt₃, more preferably SO₃-pyridine. According to the invention the ratio of such a sulfation reagent with respect to the glucose unit of the cellulose nanocrystal is preferably from 12:1 to 1:1, more preferably about 1.5:1.

This sulfation provides the CNC with controlled sulfatation and the reaction preferably occurs in presence of a solvent, more preferably the solvent being dimethylsulfoxide (DMSO) or N,N-dimethylacetamide (DMA). Much more preferably, the solvent is dimethylsulfoxide (DMSO).

The choice of the solvent was also crucial for the invention. The pristine cellulose nanocrystals are know for being sensible to the reaction temperatures, where the known techniques are usually based on warmed DMA in presence of LiCl (120 C° 1 h). On the contrary, the present invention surprisingly found the good dispersibility of the pristine cellulose nanocrystals (CNC**) in DMSO, thus allowing mild reaction conditions that are preferably at about 25° C. for 4 hours (see experimental). This is a valuable result. Thanks to the possibility of carry out the process at mild reaction conditions the present invention avoids any depolymerization side-reaction, that often occurs within the commonly used sulfation protocols that include high reaction temperature steps as, for example, in terms of temperature of 60° C.-110° C.

The addition of three fractioned batches of the SO₃-pyridine reagent, proved to be crucial for the reproducibility of the protocol and the homogeneity of the reaction mixture. The present inventors surprisingly found that this reaction is more successful if carried out in a controlled way, adding the SO₃-pyridine reagent in a fractionated schedule (i.e. three batches one of which every hour, see experimental) thus avoiding the gelification of the reaction mixture. Notably, such controlled sulfation of the CNC according to the invention does not impact the possibility to address the OH groups of the glucose of the cellulose for the further functionalization of the nanoplatform (FIG. 2 and CNC*-Au-Taurine in FIG. 6). The sulfation of the invention does not make reaction of all the primary OH groups of the cellulose but certain OH groups remain open for further functionalization (see as model sample CNC*-Au-Taurine in FIG. 6).

An advantage of the manufacturing process according to the invention is that the hydrodynamic diameter of the cellulose nanoparticles and the surface charge are not modified during the sulfation of the starting material. In more details, the diameter of the cellulose nanoparticles as starting material (pristine CNC**) is of about 70 nm and the diameter of the cellulose nanoparticles after sulfation step becoming cellulose nanoparticles with controlled sulfation (CNC*) is about 57 nm (CNC*=57 nm vs pristine CNC**=70 nm). The surface charge of the cellulose nanoparticles as starting material (pristine CNC**) is −39±2 mV and the surface charge of the cellulose nanoparticles with controlled sulfation is −45±2 mV (g-potential of CNC*=−45±2 mV vs pristine CNC** −39±2 mV). These values are considered comparable, without modifications. According to the invention, the CNC*-Au-LA was prepared using the sulfation process as described before (FIG. 2) and a significant improvement of the dispersibility and colloidal stability was obtained compared to the CNC*-Au-LA composite obtained starting from pristine CNC**, without sulfation of the cellulose nanocrystals at the beginning (FIG. 7). Therefore, the sulfation of the pristine CNC ensured the possibility to access to a scalable and modular protocol for the further functionalization of the CNC*-Au-LA and to obtain CNC*-Au-Tm conjugates (FIG. 6) with high batch-to-batch reproducibility and consequently to use of these conjugates and related compositions containing thereof in biological applications, leading to the use of the conjugates and related compositions in medicine. The functionalization is performed according to the invention through an azide-alkyne cycloaddition reaction (FIG. 3 and FIG. 5-6), eventually in presence of a catalyst that can be represented by copper. Preferably, in case copper is used as a catalyst of the reaction it is in form of CuSO₄.

The functionalization according to the invention is based on chemistry and the key features that can be listed as:
Control of the head groups loading (see CNC*-Au-Lac, FIG. 6),
Control of the functional stabilizer loading (see CNC*, FIG. 2),
Grafting of one to up to three (3) different head groups on the nanomaterial surface (CNC*-Au-Tm (where Tm=Glc or Man or Lac), CNC*-Au-BODIPY, CNC*-Au-Glc-BODIPY, FIG. 6),
Grafting of one up to two (2) different head groups by click chemistry (CNC*-Au-Glc-BODIPY, FIG. 6),
High batch-to-batch reproducibility (CNC*-Au-LA in FIG. 2 and CNC*-Au-Man in FIG. 6, see experimental),
Control of the biological identity, and
Scalable manufacturing process (FIG. 2).

According to invention it is possible to graft up to two (2) different bioactive head groups through a click chemistry on the alkyne residue, while the third bioactive headgroup can be conjugated on the cellulose's OH groups.

Such kind of achievements can be obtained owing on the unique structure and composition of the CNC*-Au-LA material according to the invention. The presence of a controlled amount of lipoamine (LA) units and the type of chemistry selected for conjugation represented by the azide-alkyne cycloaddition reaction ensures (i) a control of the head-groups loading by simply tuning the equivalent of the azide bearing reagents employed (see example of CNC*-Au-Lac and CNC*-Au-Glc, FIG. 6) and (ii) the iterative step-by-step conjugation reactions that allow to install up to two different bioactive headgroups on the same anchoring point (the alkyne residue) on the gold surface (FIG. 5-6) with a high batch-to-batch reproducibility. Conjugation reaction can be performed both in water and in polar organic solvents such as, for example, DMF, DMSO according with the solubility of the reactive headgroups.

As represented in FIG. 5 the functionalization according to the invention is carried out with the azide-alkyne cycloaddition reaction (CuAAC), leading to the monofunctional CNC*-Au conjugate that can be in turn subject to additional functionalization procedure in the same way through another azide-alkyne cycloaddition reaction (CuAAC) adding the second functionalization to give the bifunctional CNC*-Au conjugate. The headgroups of the invention are biocompatible and easily accessible mono- and disaccharide derivatives selected as targeting moiety. As above anticipated, these are preferably glucose (Glc) mannose (Man) or lactose (Lac) (FIG. 6) that are used to prove the preferential delivery of the cargos into the selected cells by the targeting of overexpressed receptors on cancer cells (i.e. GLUT channels, Mannose Receptor).

According to one embodiment of the invention, the present inventors propose the design and the use of the new ad hoc engineered material as above disclosed, as a functional and tumor specific nano-radiosensitizer aimed at the enhancement of radiation cytotoxicity at the tumor site by the targeted delivery of functionalized AuNPs stably embedded into a non-toxic and biocompatible cellulose nanocrystals (CNCs) matrix. This hybrid nanomaterial provides an efficient accumulation of small AuNPs inside tumor cells thus avoiding the fast turnover associated to small nanoparticles, thanks to the shape and the size of the CNC matrix. According to this embodiment, the targeting tumor is reached by exploiting the small biomolecules (monosaccharides) that target specific receptors expressed on cancer cells.

According to the invention, the nanomaterial as designed by the inventors is able to increase the radiation dose deposition at the tumor site thereby allowing to use lower and safer radiation doses for non-target tissues for a same tumor effect or increase the therapeutic effect while delivering the same dose for radioresistant tumors. This preserves healthy cells or tissues from being exposed generating too much side-effects. As explained the material consists the small AuNPs, functionalized on their surface with sugar headgroups as tumor targeting molecules and stably embedded into the non-toxic and biocompatible cellulose nanocrystals as above disclosed.

According to the invention, the CNC*-Au-Tm acts as a so called ‘Trojan horse’ allowing the internalization and AuNPs accumulation in the tumor cells by means of the specific targeting of glycan-binding proteins (GBP) (over)-expressed on tumor cell surface. Worth noting, the CNC carrier ensures stable AuNPs dispersions overcoming AuNPs aggregation in cell media and avoiding the fast cell turnover of small AuNPs. Compared to other approaches, the carbohydrate nature of the carrier prevents the protein corona formation, which often affects NPs efficacy thus, improving biodistribution and reducing NPs clearance. In addition, according to a preferred embodiment, some of the following major challenge in this field have been addressed and solved.

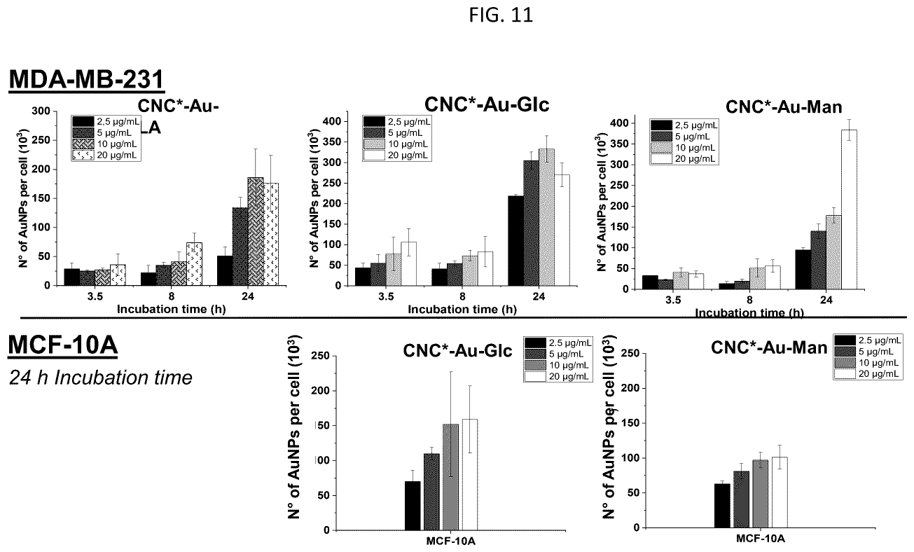

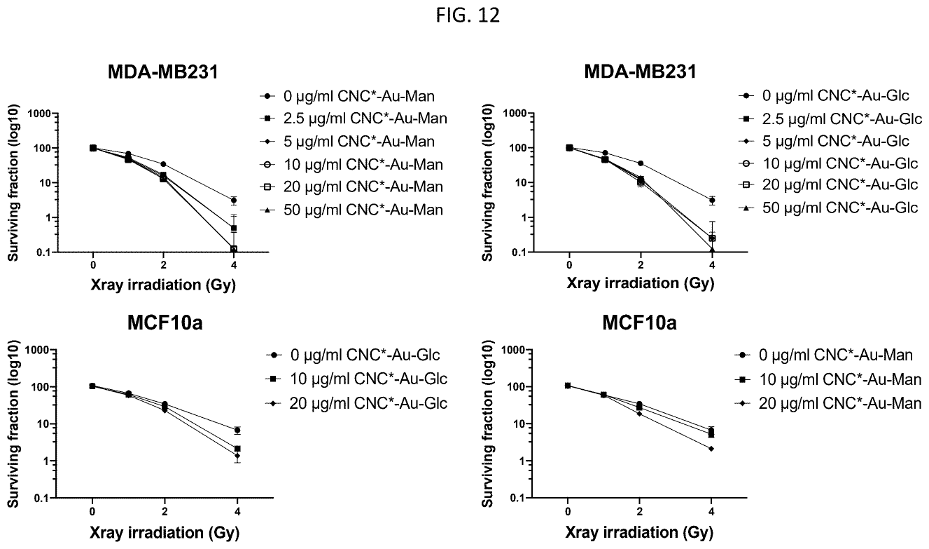

1. The amount of gold radiosensitizer employed during the treatment significantly affects translational research and limits the application into clinics. In this regard, the lower gold (Au) amount described so far ranges around 100-200 μg/mL, whereas the highest amount used according to the invention is ranging from 1.0 to 2.0 ug/mL preferably of about 20 μg/mL of the CNC-Au-Tm leading to the preferred final gold (Au) amount around 2.0 ug/mL (FIG. 11).

2. The amount of gold (Au) internalized into tumor cells, and its turnover. This parameter significantly affects the possibility of increasing the radiation dose absorption (due to the higher Z), hence, the radiation effect at the tumor site. According to the preferred embodiment of the invention the small sized gold nanoparticles (AuNPs), preferably measuring less than 5 nm (<5 nm), are promising radiation dose enhancer, although the low driving force for their uptake and their rapid renal clearance limit translation into clinics. The use of the cellulose nanocrystals (CNCs) as scaffold for the targeted delivery of the radiosensitizer into tumor cells according to the invention allows to relieve the fast turnover and to provide a preferential accumulation at the tumor site.

3. The dose enhancement factor (DEF) reached. This parameter clearly shows the efficiency of the therapeutic approach (see experimental and Table 1—FIG. 17). All the efforts reported so far in the field before the present invention such as, for example, cell cycle synchronization using liposome-based nanovectors, targeting moieties such as an example the folic acid, or specific stress conditions such as an example the cell starvation dealing with increasing the degree of gold (Au) internalization into tumor cells are known to result in an maximum amount of AuNPs uptake around 3.0-8.0×10⁴ NPs/cells.

Surprisingly, as reported in FIG. 9, the present invention results showed that the CNC*-Au-Tm composites of the invention were able to deliver to MDA-MB-231 cells, three times higher concentration of AuNPs (i.e., CNC*-Au-Man: 3.03×10⁵ NPs/cell; CNC*-Au-Glc: 3.33×10⁵ NPs/cell, FIG. 11). In addition, the presence of mannose or glucose residues in respectively CNC*-Au-Man and CNC*-Au-Glc, significantly increased the cellular uptake up of 73% and 87% respectively, compared to the non-functionalized CNC*-Au-LA (FIG. 11). Two different approaches have been used by the inventors to evaluate the biological effects of CNC*-Au-Tm on MDA-MD-231 cells. In the first assay the inventors tested the short term response of cancer cells 24 hrs post irradiation. In the second it has been evaluated the ability of irradiated cells to create colonies up to 14 days after Xrays (FIG. 12). It was observed an early arrest in proliferation in cells treated with CNC*-Au conjugates and X-Rays compared to irradiated only, most likely due to an increased radiation-related damage; furthermore, on the long term cells have lost their ability to give start to colonies. DEF measured on CNC*-Au-Tm reached uptake level are 1.83 for CNC*-Au-Glc and 1.72 for CNC*-Au-Man (FIG. 12, Table 1—FIG. 17).

Moreover, inventors tested uptake and biologic effects of CNC*-Au conjugates on MCF10a cells, an immortalized breast cell line that better resembles healthy cells. Even though it is well known that MCF10a cells have an induced proliferative behavior (30 hrs doubling time) and a general plasma membrane translocation of glucose channel 4 (GLUT4) due to the growth medium conditioning (DMEM+10% NA Fetal bovine serum+epidermal growth factor+insulin+hydrocortisone), this model is widely used to give an in vitro estimation of possible side effects of drugs and compounds on normal cells.

Compared to MDA-MB231 cells, MCF10a model shows both lower uptake levels (i.e., CNC*-Au-Man: 101×10⁵ NPs/cell; CNC*-Au-Glc: 159×10⁵ NPs/cell, FIG. 11) and lower DEF (i.e., CNC*-Au-Man: 1.04; CNC*-Au-Glc: 1.24 calculated using the concentration of 20 μg/mL of CNC*-Au conjugates, FIG. 12 and Table 1—FIG. 17) measured as previously described for MDA-MB231, thus highlighting strong selectivity of cancer to normal cells.

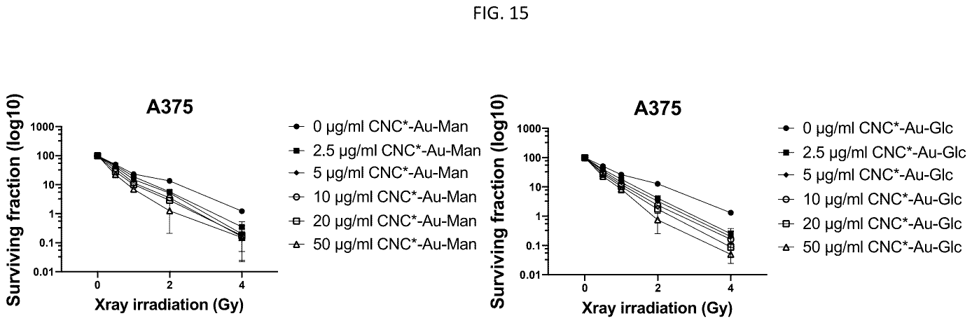

This study has been extended to skin cancer, and in particular the A375 human melanoma cell line has been selected. Inventors observed a significantly stronger cooperation between CNC*-Au conjugates and Xrays (FIG. 15 and Table 2—FIG. 18, i.e. CNC*-Au-Man: DEF 1.81; CNC*-Au-Glc: DEF 2.0). Notably, comparing A375 melanoma cell line results with the art, inventors observed a up to DEF for CNC*-Au-Glc (FIG. 15, Table 2—FIG. 18) calculated using the concentration of 20 μg/mL of CNC*-Au-Glc. This result, compared to a DEF 1.2 described in literature using untargeted AuNPs coated with PEG spacer, highlights the stronger ability of CNC*-AuN-Glc to effectively enhance Xrays effects in radiosensitive cells as to other AuNPs in the art.

According to the invention, and as said above, the CNC-Au-Tm composites are biocompatible and efficient nanovectors for the delivery of the gold radiosensitizer into the desired cells or tissues, preferably the cancer cells, as they are able to act as Trojan horse allowing the internalization and AuNPs accumulation in the desired cells or tissues, preferably tumor cells, and according to a preferred embodiment into the MDA-MB-231 breast cancer cells. Then, the cellulose nanocrystal (CNC) carrier ensures stable AuNPs dispersion overcoming AuNPs aggregation in cell media and avoiding the fast cell turnover of small AuNPs. Surprisingly, in addition to the innovation provided by the biocompatible nanomaterial of the invention, the inventors noted that it clearly indicates that the targeting of cancer glycan-binding proteins is a smart and an efficient approach, corroborating their hypothesis. In particular:

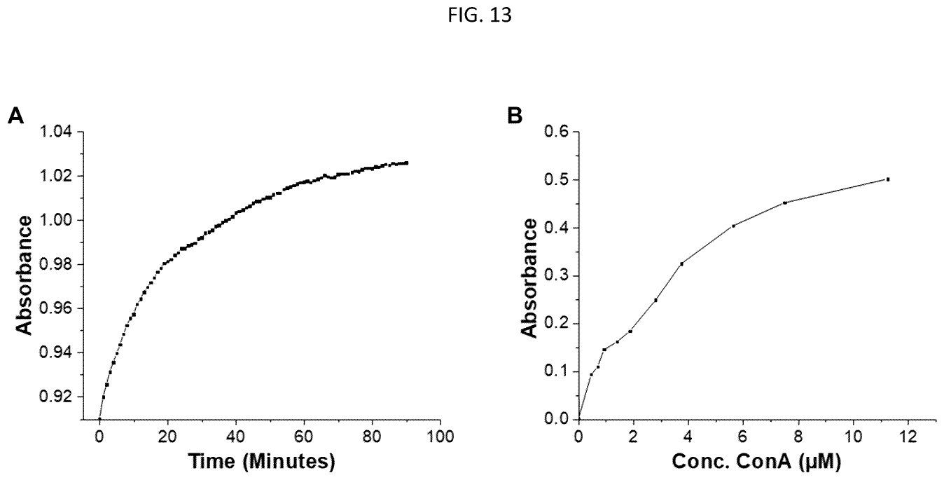

1. Robust and standardized protocol to prepare stable CNC*-Au-Tm composites (FIG. 6). A significant improvement of the CNC*-Au-Tm dispersibility and stability in diverse media (such as, for example, HEPES; DMEM; culture cells media) was addressed by increasing the sulfation degree of the starting cellulose nanocrystal as above disclosed (CNC**) The functionalization with the target molecule (Tm), such as mannose residues in the CNC*-Au-Man, was assessed by turbidimetry assays using specific plant glycan-binding lectins ensuring that the assembly of the target molecule (Tm) on the nanosystem surface allows the proper recognition by glycan-binding proteins (FIG. 13) such as Concavalin A.

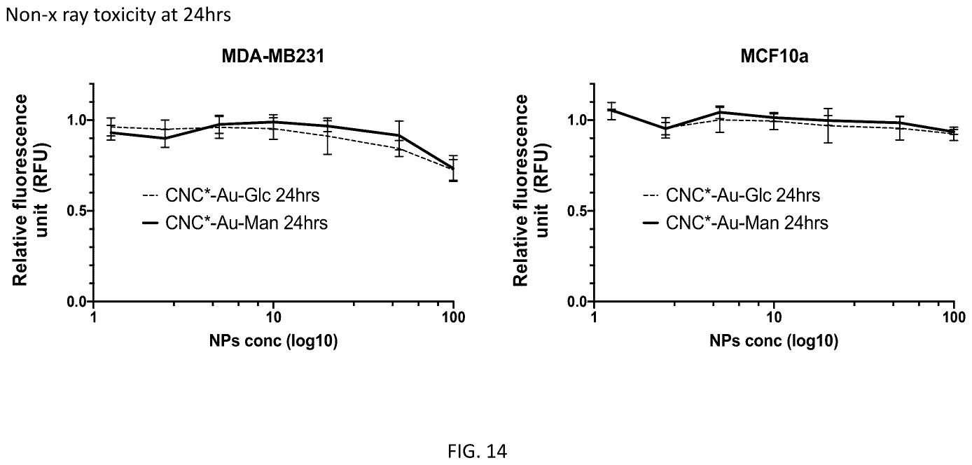

2. CNC*-Au-Tm composites showed no toxicity at relevant high concentrations (FIG. 14).

3. CNC*-Au-Tm composites efficiently act as Trojan horse significantly ensuring the uptake of the radiosensitizer in the desired cells or tissues such as for example the MDA-MB-231 breast cancer cells (FIG. 11). Results confirmed that the cellulose carrier is able to act as Trojan horse and is able to deliver high amount of AuNPs inside the desired cells such as the tumor cells. In addition, the sugar headgroups play a crucial role in the interaction with the desired cells, as the tumor cells, dramatically affecting the uptake of the nanoparticles (NPs). Indeed, both glucose and mannose residues allow obtaining a massive internalization despite using a dose of nanocomposite five time lower than the IC50.

4. The targeting of cancer glycan-binding proteins by using sugar headgroups as targeting molecules (Tm) was very advantageous, not only because it increased the specific localisation on the desired cancer cells, but it improved the uptake and retention of the material compared to non functionalised AuNPs. Moreover, the inventors observed slightly different efficiency in uptake between CNC-Au complexed with the preferred headgroups, being preferably mannose or glucose; this can give further specificity in targeting cancer cells allowing to choose the sugar headgroup that best fits tumor type according to specific glycan-binding proteins expression (FIG. 11).

5. Internalized CNC-Au-Tm increased the efficacy of radiotherapy both in the short term count (24 hrs post irradiation) and in the clonogenic ability of MDA-MB-231 cells (14 days post irradiation), showing a reduction in proliferation that highly correlates with the amount of Au measured inside cells, regardless of which sugar headgroup is present on the CNC-Au (FIG. 11 and Table 1—FIG. 17). The sugar moieties in the material of the invention are involved in targeting and it has been discovered that they have no effects per se after internalized, since quantity of the gold (Au) alone drives the radiosensitization.

Thank you to the characteristics of the hybrid cellulose nanocrystal-gold nanoparticles of the invention it is possible to obtain the maximized encapsulation of at least one drug inside the material; in particular the porosity characteristics of cellulose allows an improved encapsulation of said at least one drug, so driving locally the effect and the release of the same at a site-specific target. Drugs are embedded in the cellulose nanocrystal matrix and the bioactive head groups determine the biological activity with an increased level of versatility and reproducibility. The different bioactive head-groups determine the focus on different biological effect, that can be therefore modulated to fit the desired treatment or the desired effect to be achieved. The improved flexibility and reproducibility given by the hybrid cellulose nanocrystal-gold nanoparticles of the invention leads to a significant advantage, preferably in medicine field and more preferably in the treatment of tumors. The presence of these bioactive head-groups represents a significant stride over the known materials, and the obtainment of an improved flexibility and versatility met the need of this technical field, representing the progress.

Another advantage of the hybrid cellulose nanocrystal-gold nanoparticles of the invention is represented by the selectivity given by the functionalization of the material; as above disclosed, the modulation of the biological effect is focused on the possibility to drive the biological effect on the target cells, avoiding the healthy cells or the healthy tissues only looking for the pathological target. Such selectivity is related to the selection of the targeting molecule and to the desired treatment.

Thanks to the hybrid cellulose nanocrystal-gold nanoparticles of the invention the effect on the healthy cells or healthy tissue has been significantly reduced or cancelled, treating only where necessary especially treating tumor cells not healthy cells. According to the preferred embodiments, tumor cells demonstrate a significant response to the treatment according to the invention, while the healthy cells are not interested.

Another advantage is the possibility to the stoichiometrical control of the reactions in the manufacturing process. Accordingly, the speed of the reaction is not controlled but through the stoichiometry of the reagents (see examples of CNC*-Au-Lac and FIG. 6) of the invention it is advantageously possible not to saturate all the alkyne residues making reaction only with part of them with the first bioactive headgroup and then the remaining ones with the second headgroup.

According to the above, it should be evidenced that the hybrid cellulose nanocrystal-gold nanoparticles of the invention are a new and improved solution for the nanotechnology in medicine: controlled and more efficient delivery and release of therapeutic molecules associated with longer half-life of drugs within the body and reduced toxicity of treatment is the gold that the inventors has surprisingly reached, preferably directed to the treatment of tumor, preferably solid tumors and even more preferably focused to the radiotherapy with gold particles.

### EXAMPLES

### Synthesis of CNC*-Au-Tm.

CNC*-Au conjugates where bioactive headgroups are mono- and disaccharide derivatives have been prepared (FIG. 6). Mono- and disaccharide derivatives correspond to tumor targeting moeities (named Tm). Accordingly, Tm have been selected among glucose (Glc), mannose (Man), lactose (Lac) glycosides bearing a triethylene pegylated spacer at the anomeric position of respectively a D-glucose or a D-Mannose molecule and of the D-glucose residue of the Lactose molecule. The PEG spacer contains a terminal azide group (Azido-PEG-Tm). The terminal azide group was reacted with the terminal alkyne group of the LA installed on Au surface of the CNC*-Au-LA (FIG. 6) by Cu-catalyzed azide-alkyne reaction in water. According with the equivalent of the reactive azide derivatives the loading of the bioactive headgroups can be controlled. CNC*-Au conjugates have been easily purified by dialysis vs milliQ water.

a) Assessment of the high batch-to-batch reproducibility of the functionalization step. In particular, in order to asses the high batch-to-batch reproducibility of the functionalization step the azido-PEG-Man was selected as model Tm. Click reactions using two equivalents (respect to LA on CNC*-Au-LA surface) of azido-PEG-Man (FIG. 6) were repeated in triplicates. Accordingly three conjugates of CNC*-Au-Man with a loading of the azido-PEG-Man of 0.0154±0.001 mmol/100 mg (based on N content from elemental analysis) were obtained. This conjugate was used in the cell uptake assay (FIG. 11) and in the colony forming assay (FIG. 12).

The same protocol was employed for the preparation of CNC*-Au-Glc (FIG. 6) using the azido-PEG-Glc as azide bearing reagent. The CNC*-Au-Glc with an azido-PEG-Glc loading of 0.0162 mmol/100 mg (based on N content from elemental analysis) using respectively 2.0 equivalents (respect to LA on CNC*-Au-LA surface) of the azido-PEG-Glc has been obtained. This conjugate was used in the cell uptake assay (FIG. 11) and in the colony forming assay (FIG. 12).

b) Assessment of the control of the loading of the bioactive headgroups.

CNC*-Au conjugates with a control of the loading of the sugar headgroup have been prepared. In particular, azido-PEG-Lac has been selected as model bioactive headgroup (FIG. 6). Three conjugates bearing lactose residues with an azido-PEG-Lac loading of 0.0126 mmol/100 mg, or 0.0104 mmol/100 mg, or 0.00738 mmol/100 mg, using respectively 1.5 or 0.5 or 0.15 equivalents (respect to LA on CNC*-Au-LA surface) of azido-PEG-Lac have bee prepared.

The same protocol was employed for the preparation of CNC*-Au-Glc with an azido-PEG-Glc loading of 0.0088 mmol/100 mg (based on N content from elemental analysis) using 0.3 equivalents (respect to LA on CNC*-Au-LA surface) of azido-PEG-Glc. This conjugate was used to prove the possibility of grafting up to two (2) different head groups by click chemistry (see below).

### Synthesis of CNC*-Au-BODIPY.

A CNC*-Au conjugate where the bioactive headgroup is a fluorescent probe has been prepared. The azide-bearing tetramethyl-BODIPY (FIG. 6) was selected as model fluorescent probe and it was reacted with CNC*-Au-LA using 0.1 equivalents of BODIPY (respect to LA on CNC*-Au-LA surface). The functionalization was assessed by the emission of fluorescence of the conjugate CNC*-Au-BODIPY (λₘₐₓ=530 nm).

### Synthesis of CNC*-Au-Glc-BODIPY.

A CNC*-Au conjugate where up to two (2) different head groups are included on nanomaterial surface by click chemistry, has been prepared. In particular, Glucose and BODIPY have been selected as model headgroups. The CNC*-Au-Glc-BODIPY (FIG. 6) was prepared by means of step-by-step iterative CuACC reactions (FIG. 5). A control of the headgroups loading has been reached by modulating the equivalents of the azide bearing reagents. At first, the CNC*-Au-LA was reacted with 0.3 equivalents of azido-PEG-Glc (respect to LA on CNC*-Au-LA surface) as described before (FIG. 6). Then, the CNC-Au-Glc conjugate was reacted with 0.5 equivalents (respect to LA on CNC*-Au-LA surface) of the azide-bearing BODIPY (FIG. 6) affording the CNC*-Au-Glc-BODIPY. The amount of BODIPY was estimated was quantified by UV-vis spectroscopy using the molar extinction coefficient of the BODIPY in DMSO.

### Synthesis of CNC*-Au-LA-Taurine and CNC*-Au-LA-Taurine-BODIPY.

As mentioned before, the controlled sulfation of the CNC according to the invention does not impact the possibility to address the OH groups of the glucose of the cellulose for the further functionalization of the nanoplatform. Indeed, the sulfation of the invention does not make reaction of all the primary OH groups of the cellulose but certain OH groups remain open for further functionalization. This is advantageously discovered by the present inventors, differently to what is known in the art such as for example in the CN111803629A where this is not possible. In this regards, the 2-aminoethanesulfonic acid (Taurine, FIG. 6) was selected as model headgroup and it was reacted with the CNC*-Au-LA. In particular, the OH groups of the Glc residues of the CNC*-Au-LA were reacted with 1,1′-Carbonyldiimidazole (CDI) to introduce carboxyl moieties for further extension with the Taurine. The reaction proceeds by the formation of reactive imidazole carbamate intermediate, which in turn reacts with the Taurine leading to a stable urethane linkage (CNC*-Au-LA-Taurine). Then, the possibility to still include a bioactive headgroup on gold surface was assessed. Indeed, the resulting CNC*-Au-LA-Taurine was further functionalized and BODIPY has been selected as model headgroup. Then, the CNC*-Au-LA-Taurine conjugate was reacted with 0.5 equivalents (respect to LA on CNC*-Au-LA surface) of the azide-bearing BODIPY (FIG. 6) affording the CNC*-Au-LA-Taurine-BODIPY. The amount of BODIPY was estimated was quantified by UV-vis spectroscopy using the molar extinction coefficient of the BODIPY in DMSO.

### EXPERIMENTAL PART

### Synthesis of CNC*-Au Conjugates (FIG. 5-6).

### Synthesis of CNC*(FIG. 2).

CNC** (1.0 g, 5.5 mmol of glucose units) was added to vigorously stirred DMSO (21 mL) at 25° C., the dispersion was stirred for 30 minutes then SO₃-pyridine complex (400 mg, 2.51 mmol) was added, and the dispersion diluted with DMSO (10.5 mL) to avoid gelification. Two additional batches of sulfur trioxide pyridine complex (400 mg, 2.51 mmol and 500 mg, 3.14 mmol respectively) were added after respectively 1 h and 2 h. The reaction mixture was stirred for additional 2 h (total reaction time 4 h) then it was diluted with acetone (100 mL) and the CNC* was precipitated by centrifugation (8000 rpm, 10 minutes). The supernatant was removed and the solid dispersed in acetone (100 mL) and precipitated by centrifugation (8000 rpm, 10 minutes). The solid was recovered and dispersed in a solution of NaOH (40 mL, 0.1 M in H₂O) and stirred for 30 minutes. The dispersion was diluted with milliQ water (60 mL) and dialyzed vs milliQ water for 72 h. Then, the dispersion was lyophilized to obtain the CNC* as a fluffy white solid. Elemental analysis C, 38.44%, H, 6.18%, N, 0.01% and S, 1.99%. The reaction was repeated in triplicate affording a degree of sulfation (DS=0.114±0.007).

### Synthesis of CNC*-Au-LA (FIG. 2).

A stirred solution of tetrachloroauric acid trihydrate 80 mg (0.23 mmol) in milliQ water (23 mL) was diluted with methanol (207 mL), then 230 mg of CNC* were added, and the mixture vigorously stirred for 30 minutes. Then, a solution of LA (172 mg, 0.71 mmol) in methanol (1.5 mL) and sodium borohydride (196 mg, 37.8 mmol) were added sequentially and the mixture stirred for 2 h at r.t. Finally, the CNC*-Au-LA dispersion was dialyzed for 24 h vs milliQ water and freeze dried to afford 385 mg of CNC*-Au-LA. ICP-AES: Au 9.9% w/w. Elemental analysis C, 30.64%, H, 5.47%, N, 0.73% and S, 3.21%. The reaction was repeated in triplicate affording a LA loading of 0.046±0.003 mmol/100 mg (based on N content from elemental analysis).

### Synthesis of CNC*-Au-Man (FIG. 5-6).

To a stirred solution of CNC*-Au-LA (60 mg, 0.038 mmol of LA) in milliQ water (6.0 mL) azido-PEG-Man (26 mg, 0.076 mmol) was added and the mixture stirred for 30 minutes. Then, 110 μL of a 50 mM solution of a mixture CuSO₄:sodium ascorbate in a 1:1 ratio were added (0.0055 mmol). The reaction mixture was stirred at r.t. for 5 h. Then, an additional batch of catalyst was added (110 uL of 50 mM stock solution, 0.0055 mmol) and the mixture stirred at r.t. for 12 h. Then, the reaction mixture was dialyzed vs milliQ water for 24 h and freeze dried to afford 58 mg of CNC*-Au-Man. ICP-AES: Au 9.9±% w/w. Elemental analysis C, 36.68±0.06%, H, 6.12±0.11%, N, 1.25±0.04% and S, 3.21±0.07%. The reaction was repeated in triplicate affording an Azido-PEG-Man loading 0.0154±0.001 mmol/100 mg (based on N content from elemental analysis).

### Synthesis of CNC*-Au-Glc (2.0 eq of Azido-PEG-Glc, FIG. 5-6).

To a stirred solution of CNC*-Au-LA (60 mg, 0.038 mmol of LA) in milliQ water (6.0 mL) azido-PEG-Glc (26 mg, 0.076 mmol) was added and the mixture stirred for 30 minutes. Then, 110 uL of a 50 mM solution of a mixture CuSO₄:sodium ascorbate in a 1:1 ratio were added (0.0055 mmol). The reaction mixture was stirred at r.t. for 2 h. Then, an additional batch of catalyst was added (110 uL of 50 mM stock solution, 0.0055 mmol) and the mixture stirred at r.t. for 12 h. Then, the reaction mixture was dialyzed vs milliQ water for 24 h and freeze dried to afford 56 mg of CNC*-Au-Glc. ICP-AES: Au 10.6% w/w. Elemental analysis C, 36.45%, H, 5.59%, N, 1.28% and S, 4.74%. Azido-PEG-Glc loading 0.0162 mmol/100 mg (based on N content from elemental analysis).

Synthesis of CNC*-Au-Glc (0.3 eq of azido-PEG-Glc, FIG. 5-6).

To a stirred solution of CNC*-Au-LA (100 mg, 0,047 mmol of LA) in milliQ water (10 mL) azido-PEG-Glc (4.8 mg, 0,014 mmol) was added and the mixture stirred for 30 minutes. Then, 141 uL of a 50 mM solution of a mixture CuSO₄:sodium ascorbate in a 1:1 ratio were added (0.0141 mmol). The reaction mixture was stirred at r.t. for 2 h. Then, an additional batch of catalyst was added (141 uL of 50 mM stock solution, 0.0141 mmol) and the mixture stirred at r.t. for 12 h. Then, the reaction mixture was dialyzed vs milliQ water for 24 h and freeze dried to afford 85 mg of CNC*-Au-Glc. ICP-AES: Au 9.5% w/w. Elemental analysis C, 35.27%, H, 5.48%, N, 1.04% and S, 4.32%. Azido-PEG-Glc loading 0.0088 mmol/100 mg (based on N content from elemental analysis).

### Synthesis of CNC*-Au-Lac (1.5 eq of Azido-PEG-Lac, FIG. 5-6).

To a stirred solution of CNC*-Au-LA (50 mg, 0.025 mmol of LA) in milliQ water (5.0 mL) azido-PEG-Lac (18.71 mg, 0.0375 mmol) was added and the mixture stirred for 30 minutes. Then, 75 μL of a 50 mM solution of a mixture CuSO₄:sodium ascorbate in a 1:1 ratio were added (0.00375 mmol). The reaction mixture was stirred at r.t. for 2 h. Then, an additional batch of catalyst was added (75 uL of 50 mM stock solution, 0.0375 mmol) and the mixture stirred at r.t. for 12 h. Then, the reaction mixture was dialyzed vs milliQ water for 24 h and freeze dried to afford 45 mg of CNC*-Au-Lac. ICP-AES: Au 10% w/w. Elemental analysis C, 36.53%, H, 5.62%, N, 1.26% and S, 5.23%. Azido-PEG-Lac loading 0.0126 mmol/100 mg (based on N content from elemental analysis).

Synthesis of CNC*-Au-Lac (0.5 eq of azido-PEG-Lac, FIG. 5-6).

To a stirred solution of CNC*-Au-LA (50 mg, 0.025 mmol of LA) in milliQ water (5.0 mL) azido-PEG-Lac (6.23 mg, 0.0125 mmol) was added and the mixture stirred for 30 minutes. Then, 75 uL of a 50 mM solution of a mixture CuSO₄:sodium ascorbate in a 1:1 ratio were added (0.00375 mmol). The reaction mixture was stirred at r.t. for 2 h. Then, an additional batch of catalyst was added (75 uL of 50 mM stock solution, 0.00375 mmol) and the mixture stirred at r.t. for 12 h. Then the reaction mixture was dialyzed vs milliQ water for 24 h and freeze dried to afford 45 mg of CNC*-Au-Lac. ICP-AES: Au 10.6% w/w. Elemental analysis C, 36.35%, H, 5.42%, N, 1.17% and S, 5.14%. Azido-PEG-Lac loading 0.0104 mmol/100 mg (based on N content from elemental analysis).

Synthesis of CNC*-Au-Lac (0.15 eq of azido-PEG-Lac, FIG. 5-6).

To a stirred solution of CNC*-Au-LA (50 mg, 0.025 mmol of LA) in milliQ water (5.0 mL) azido-PEG-Lac (6.23 mg, 0.0025 mmol) was added and the mixture stirred for 30 minutes. Then, 75 uL of a 50 mM solution of a mixture CuSO₄:sodium ascorbate in a 1:1 ratio were added (0.00375 mmol). The reaction mixture was stirred at r.t. for 2 h. Then, an additional batch of catalyst was added (75 uL of 50 mM stock solution, 0.00375 mmol) and the mixture stirred at r.t. for 12 h. Then, the reaction mixture was dialyzed vs milliQ water for 24 h and freeze dried to afford 45 mg of CNC*-Au-Lac. ICP-AES: Au 10.7% w/w. Elemental analysis C, 36.17%, H, 5.50%, N, 1.04% and S, 4.98%. Azido-PEG-Lac loading 0.00738 mmol/100 mg (based on N content from elemental analysis).

### Synthesis of CNC*-Au-BODIPY (FIG. 5-6).

To a stirred solution of CNC*-Au-LA (30 mg, 0.014 mmol of LA) in dimethyl formamide (3.0 mL) azido-tetramethyl-BODIPY (510 μg, 1.4 μmol) and the mixture stirred for 30 minutes. Then, 42 uL of a 50 mM solution of a mixture CuSO₄:sodium ascorbate in a 1:1 ratio were added (0.0021 mmol). The reaction mixture was stirred at r.t. for 2 h. Then, an additional batch of catalyst was added (42 uL of 50 mM stock solution, 0.0021 mmol) and the mixture stirred at r.t. for 12 h. Then, the mixture was diluted with dichloromethane and centrifuged (9000 rpm, 10 minutes). The solid was recovered, dispersed in 10 mL of methanol and precipitated by centrifugation (9000 rpm, 10 minutes). The solid was collected, dispersed at 1.0 mg/mL in a mixture 9:1 water:methanol and dialyzed for 48 h vs a mixture 9:1 water:methanol (replacing the solution two times) and 24 h vs water. The dispersion was then freeze dried to afford the CNC*-Au-BODIPY (25 mg) that provides fluorescence emission at λₘₐₓ=530 nm.

### Synthesis of CNC*-Au-LA-Taurine (FIG. 5-6).

To stirred dispersion of CNC*-Au-LA (45 mg) in dry Dimethyl sulfoxide (2.5 mL) under nitrogen atmosphere was added carbonyl diimidazole (81 mg, 0.5 mmol). The mixture was stirred for 3 h at r.t., then taurine (250 mg, 2 mmol) was added and the mixture was stirred under nitrogen atmosphere at r.t. for 11.5 h. Then sodium carbonate anhydrous (53 mg, 0.5 mmol) was added and the mixture stirred for another 30 minutes. the dispersion was diluted with milliQ water (47.5 mL) and dialyzed vs water for 24 h (replacing the water three times) and freeze dried to afford 38 mg of CNC*-Au-LA-Taurine. ICP-AES Au 9.2% w/w. Elemental analysis C, 34.55%, H, 5.482%, N, 1.2%, 4.518%. Taurine loading 0.033 mmol/100 mg (based on N content from elemental analysis).

### Synthesis of CNC*-Au-LA-Taurine-BODIPY (FIG. 5-6).

To a stirred solution of CNC*-Au-La-Taurine (20 mg, 0.010 mmol of LA) in dimethyl formamide (2.0 mL) azido-tetramethyl-BODIPY (1.9 mg, 5 μmol) and the mixture stirred for 30 minutes. Then, 30 uL of a 50 mM solution of a mixture CuSO₄:sodium ascorbate in a 1:1 ratio were added (0.0015 mmol). The reaction mixture was stirred at r.t. for 2 h. Then, an additional batch of catalyst was added (30 uL of 50 mM stock solution, 0.0015 mmol) and the mixture stirred at r.t. for 12 h. Then, the mixture was diluted with dichloromethane and centrifuged (9000 rpm, 10 minutes). The solid was recovered, dispersed in 25 mL of methanol and precipitated by centrifugation (9000 rpm, 10 minutes) four times. The solid was collected, dispersed at 1.0 mg/mL in a mixture 9:1 water:methanol and dialyzed for 48 h vs a mixture 9:1 water:methanol (replacing the solution two times) and 24 h vs water. The dispersion was then freeze dried to afford the CNC*-Au-Taurine-BODIPY (20 mg) that provides fluorescence emission at λₘₐₓ=530 nm. The amount of BODIPY was estimated was quantified by UV-vis spectroscopy using the molar extinction coefficient of the BODIPY in DMSO. It results on 0.47% w/w of BODIPY.

### Synthesis of CNC*-Au-Glc-BODIPY (FIG. 5-6).

To a stirred solution of CNC*-Au-Glc (25 mg, 0.012 mmol of LA) in dimethyl formamide (2.5 mL) azido-tetramethyl-BODIPY (3.5 mg, 9.5 μmol) and the mixture stirred for 30 minutes. Then, 57 uL of a 50 mM solution of a mixture CuSO₄:sodium ascorbate in a 1:1 ratio were added (0.0029 mmol). The reaction mixture was stirred at r.t. for 2 h. Then, an additional batch of catalyst was added (57 uL of 50 mM stock solution, 0.0029 mmol) and the mixture stirred at r.t. for 12 h. Then, the mixture was diluted with dichloromethane and centrifuged (9000 rpm, 10 minutes). The solid was recovered, dispersed in 10 mL of methanol and precipitated by centrifugation (9000 rpm, 10 minutes). The solid was collected, dispersed at 1.0 mg/mL in a mixture 9:1 water:methanol and dialyzed for 48 h vs a mixture 9:1 water:methanol (replacing the solution two times) and 24 h vs water. The dispersion was then freeze dried to afford the CNC*-Au-BODIPY (25 mg) that provides fluorescence emission at λₘₐₓ=530 nm. The amount of BODIPY was estimated was quantified by UV-vis spectroscopy using the molar extinction coefficient of the BODIPY in DMSO. It results on 1.56% w/w of BODIPY.

### Synthesis of CNC**-Au Conjugates (FIG. 8).

### Synthesis of CNC**-Au-LA (FIG. 8)

A stirred solution of tetrachloroauric acid trihydrate 80 mg (0.23 mmol) in milliQ water (23 mL) was diluted with methanol (207 mL), then 230 mg of CNC** were added, and the mixture vigorously stirred for 30 minutes. Then, a solution of LA (172 mg, 0.71 mmol) in methanol (1.5 mL) and sodium borohydride (196 mg, 37.8 mmol) were added sequentially and the mixture stirred for 2 h at r.t. Finally, the CNC**-Au-LA solution was dialyzed (a precipitate is formed in the dialysis sack) for 24 h vs water milliQ and freeze dried to afford 392 mg of CNC**-Au-LA. ICP-AES: Au 9.18% w/w. Reaction was repeated two time affording two different batches of Batch A: elemental analysis C, 31.38%, H, 5.26%, N, 1.02% and S, 4.86%. LA loading of 0.0720 mmol/100 mg (based on N content from elemental analysis) and of Batch B: elemental analysis C, 27.76%, H, 5.10%, N, 0.95% and S, 4.08%. LA loading of 0.067 mmol/100 mg (based on N content from elemental analysis).

### Synthesis of CNC**-Au-Man (FIG. 8)

To a stirred solution of CNC**-Au-LA (30 mg, 0.021 mmol of LA) in milliQ water (3.0 mL) azido-PEG-Man (14 mg, 0.042 mmol) was added and the mixture stirred for 30 minutes. Then, 63 uL of a 50 mM solution of a mixture CuSO₄:sodium ascorbate in a 1:1 ratio were added (0.0031 mmol). The reaction mixture was stirred at r.t. for 2 h. Then, an additional batch of catalyst was added (63 uL of 50 mM stock solution, 0.0031 mmol) and the mixture stirred at r.t. for 12 h. Then, the mixture was dialyzed vs milliQ water for 24 h (a precipitate is formed in the dialysis sack) and freeze dried to afford 29 mg of CNC**-Au-Man. ICP-AES: Au 9.5% w/w. Elemental analysis C, 36.06%, H, 5.39%, N, 1.60% and S, 4.96%. Azido-PEG-Man loading of 0.0155 mmol/100 mg (based on N content from elemental analysis).

### Synthesis of CNC**-Au-Fuc (FIG. 8).

To a stirred solution of CNC**-Au-LA (67 mg, 0.048 mmol) in milliQ water (6.7 mL) azido-PEG-Fuc (31 mg, 0.096 mmol) was added and the mixture stirred for 30 minutes. Then, 144 uL of a 50 mM solution of a mixture CuSO₄:sodium ascorbate in a 1:1 ratio were added (0.0072 mmol). The reaction mixture was stirred at r.t. for 2 h. Then, an additional batch of catalyst was added (144 uL of 50 mM stock solution, 0.0072 mmol) and the mixture stirred at r.t. for 12 h. Then, the mixture was dialyzed vs milliQ water for 24 h (a precipitate is formed in the dialysis sack) and freeze dried to afford 27 mg of CNC**-Au-Fuc. ICP-AES: Au 10.7% w/w. Elemental analysis C, 35.02%, H, 4.55%, N, 1.58% and S, 5.7%. Azido-PEG-Fuc loading of 0.0132 mmol/100 mg (based on N content from elemental analysis).

### Synthesis of CNC**-Au-Glc (FIG. 8)

To a stirred solution of CNC**-Au-LA (30 mg, 0.021 mmol) in milliQ water (3.0 mL) azido-PEG-Glc (14 mg, 0.042 mmol) was added and the mixture stirred for 30 minutes. Then, 12 uL of a 50 mM solution of a mixture CuSO₄:sodium ascorbate in a 1:1 ratio were added (0.060 mmol). The reaction mixture was stirred at r.t. for 2 h. Then, an additional batch of catalyst was added (12 uL of 50 mM stock solution, 0.060 mmol) and the mixture stirred at r.t. for 12 h. Then, the mixture was dialyzed vs milliQ water for 24 h (a precipitate is formed in the dialysis sack) and freeze dried to afford 27 mg of CNC**-Au-Glc. ICP-AES: Au 9.8% w/w. Elemental analysis C, 30.47%, H, 4.34%, N, 2.21% and S, 4.26%. Azido-PEG-Glc loading of 0.008 mmol/100 mg (based on N content from elemental analysis).

### Resuspension of the CNC*-Au Conjugates.

The day before to experiment, powder preparation of CNC*-Au conjugates has been first vortexed 2 min, milliQ distilled water has been added dropwise while vortexing to reach 5.0 mg/ml final concentration. 5.0 mg/ml stock vials are then sonicated 30 cycles (60 sec on, 5 sec off) at 4° C. in a Bioruptor sonication bath (Biosense), after sonication, vials are left overnight to stabilize.

On the day of the experiment, 5.0 mg/ml stocks are diluted to 1.0 mg/ml adding milliQ distilled water and further sonicated 30 cycles (60 sec on, 5 sec off) at 4° C. 1.0 mg/ml vials have been added to cells growth medium to reach desired final concentrations.

### Cytotoxicity Assay (FIG. 14 and FIG. 17)

MDA-MB-231, MCF10A and A375 cells have been plated at 5 k/plate in 96 well plates, left to attach overnight. At t0 cells have been treated with CNC*-Au conjugates functionalized with either glucose or mannose at different concentrations (0, 2.5, 5, 10, 20, 50 and 100 μg/ml) for 24 hrs, in a final volume of 100 μl. After CNC*-Au conjugates incubation, viability has been assessed by cell titer glo (PROMEGA) as manufacturer's protocol, values of luminescence have been normalized against untreated controls.

### Short Term Assay

MDA-MB-231, MCF10A and A375 cells have been plated at 200 k/plate in 6 cm plates, left to attach overnight. At t0 cells have been treated with CNC*-Au-Glc and CNC*-Au-Man functionalized with respectively glucose or mannose at different concentrations (2.5, 5, 10, and 20 μg/ml or left untreated) for 24 hrs, in a final volume of 2.0 ml. After CNC*-Au-Tm conjugates incubation cells have been Xray irradiated with Faxitron CP-160 (Faxitron Xray corp) at 100 kV and 10 mA to obtain a final irradiation of 0, 0.5, 1, 2, 4 and 8 Gray in the radiation only controls and 0, 0.5, 1, 2 and 4 Gray in the CNC*-Au-Tm treated points. 24 hrs post irradiation, cells have been collected in trypsin and PI exclusion assay have been performed to count and assess viability, using MACSQuant X flow cytometer (Miltenyi Biotech). Debris have been excluded by size (forward scattering) and granularity (side scattering) evaluation.

Final data are collected from 3 independent experiments with 3 replicates each (n=9). Values are normalized against no-CNC*-Au-Tm, no-radiation control cells.

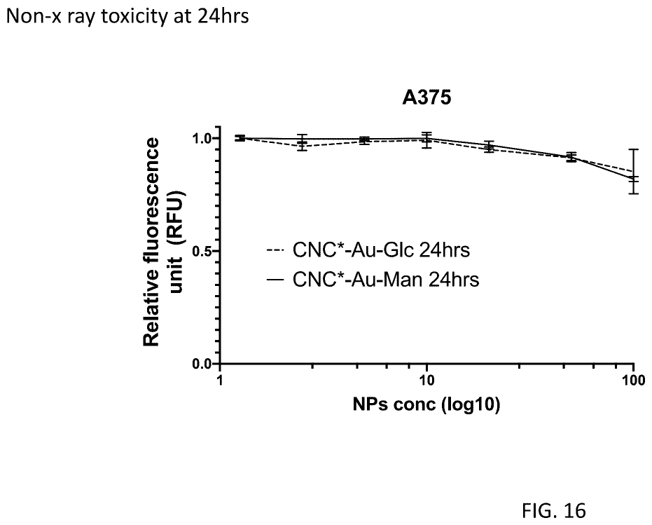

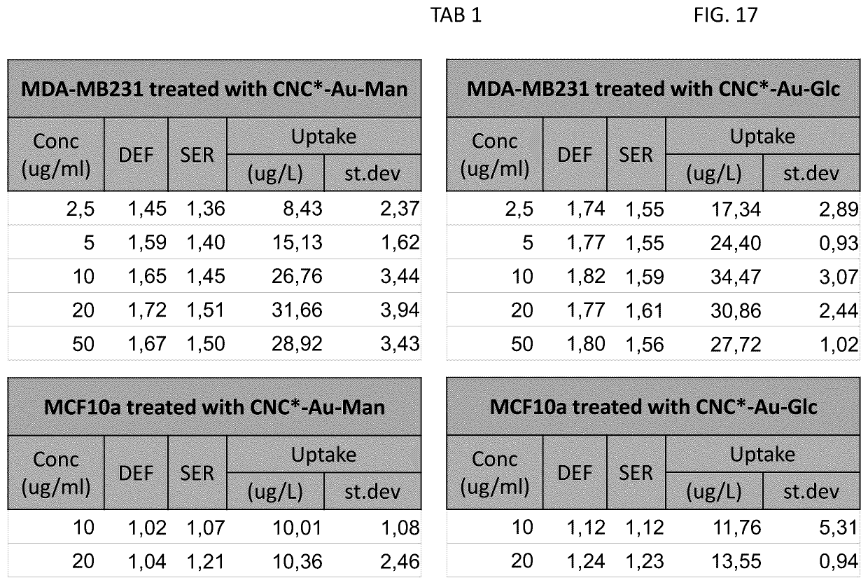

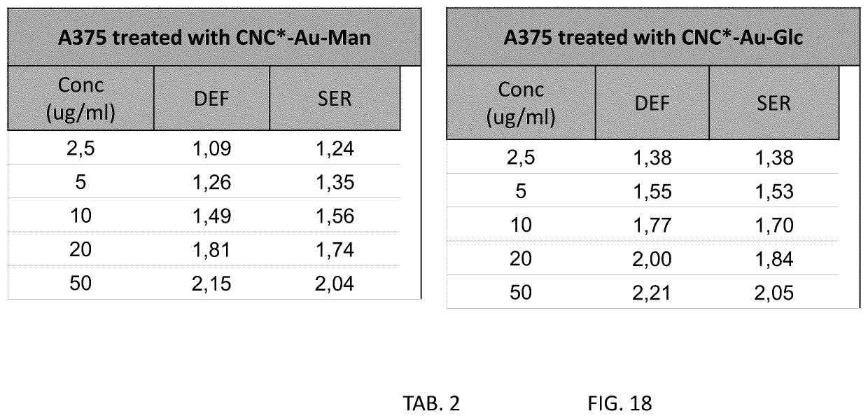

### Colony Forming Assay (FIG. 12 and FIG. 16)

MDA-MB-231, MCF10A and A375 cells have been plated at 200 k/plate in 6 cm plates, left to attach overnight. At t0 cells have been treated with CNC*-Au-Glc and CNC*-Au-Man functionalized with respectively glucose or mannose at different concentrations (2.5, 5, 10, 20 and 50 μg/ml or left untreated) for 24 hrs, in a final volume of 2.0 ml. After CNC*-Au-Tm incubation cells have been Xray irradiated with Faxitron CP-160 (Faxitron Xray corp) at 100 kV and 10 mA to obtain a final irradiation of 0, 1, 2 and 4 Gray. Minutes after irradiation, cells have been counted and re-plated at 2 density in 6 wells plates (100 and 200 cells-well) and left to grow up to 14 days. After, colonies have been stained with crystal violet and counted under microscope, considering valid colonies with more than 50 cells. Surviving fraction has been calculated as the ratio between counted colonies and plated cells.

### Methods for DEF/SER Calculation (Table 1—FIG. 17 and Table 2—FIG. 18)

DEF has been calculated from clonogenic assay, using PRISM software to first estimate non linear regression—linear quadratic model with equation Y=100*exp(−1*(A*X+B*X\hat{ }2)) for each type of CNC*-AuNPs at each concentration in each cell line. Then DEF has measured as the ratio between Xray doses required to give the same surviving fraction of 60% in CNC*-Au-NPs various conditions and in radiation only control cells.

$$Formula DEF = ( Xray dose at 60 \% surviving fraction in irradiated plus NPs ) / ( Xray dose at 60 \% surviving fraction in irradiated only )$$

SER has been calculated from clonogenic assay, using PRISM software, defined as the ratio between the area under the curve (Y=surviving fraction X=Xray increasing dosage) of irradiated plus CNC*-Au at one specific concentration and irradiated only.

$$Formula SER = ( area under the curve of cells irradiated plus NPs ) / ( area under the curve of cells irradiated only )$$

### Cell Uptake Evaluation (FIG. 11 and FIG. 15).

MDA-MB-231, MCF10A and A375 cells have been plated at 40 k/well in 24 well plates, left to attach overnight. At t0 cells have been treated with CNC*-Au-Glc and CNC*-Au-Man functionalized with respectively glucose or mannose and CNC*-Au-LA at different concentrations (2.5, 5, 10, 20 and 50 μg/ml or left untreated) for 24 hrs, in a final volume of 300 μl. After CNC*-Au incubation cells have been washed 3 times with 100 μl of PBS, then detached in 100 μl of trypsin EDTA solution and collected. MilliQ distilled water has been added to detached cells in trypsin with vigorous pipetting to cause cell lysis due to ipotonic buffer. Lysate can be stored at 20° C. 400 μl of acqua regia are added on the lysates and left 72 hrs under chemical hood. After the incubation 1500 μl of milliQ distilled water has been added and mixed by gently rocking the vials.

### Turbidimetry Assay (FIG. 13).

Turbidimetry measurements were performed in triplicate using a 96 well plate and a biorad plate reader. ConA was dissolved at the concentration of 20 μM in 10 mM HEPES (pH 7.4), 1 mM MnCl₂ and CNC*-Au-Man were dispersed at the concentration of 0.1% in milliQ water. Then the two solutions were mixed in the 96 well plate to obtain 200 μL of 10 μM of ConA and 0.01% of CNC*-Au-Man using 10 mM HEPES (pH 7.4), 1 mM MnCl₂ for the dilution. The absorbance at 490 nm was read every minute for 30 minutes.

In a second essay to determine the amount of ConA necessary to observe the agglutination of nanoparticles, CNC*-Au-Man 0.01% w/v were titrated with ConA in the in the range of concentration from 5 to 10000 nM measuring the absorbance at 490 nm after 10 minutes.

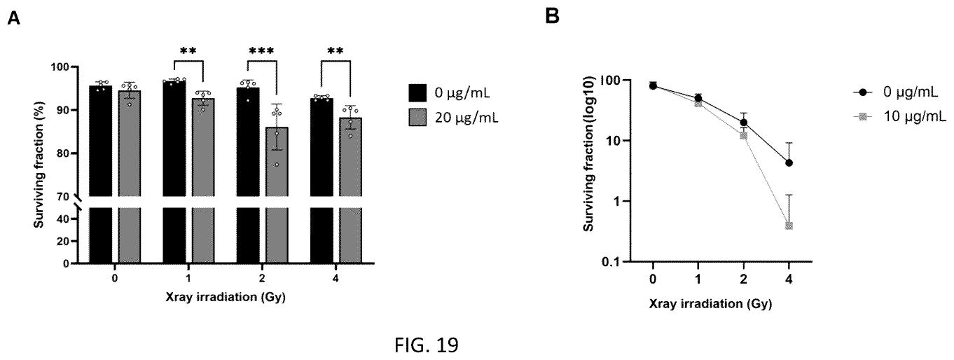

### Flow Cytometric Analysis on Treated Glioblastoma Cells (FIG. 19A)

U251 cells have been plated at 100 k/plate in 6-well plates, left to attach overnight. At t0 cells have been treated with CNC*-Au-Glc functionalized with glucose at 20 g/ml or left untreated for 24 hrs, in a final volume of 2.0 ml. After CNC*-Au-Tm incubation cells have been Xray irradiated with Faxitron CP-160 (Faxitron Xray corp) at 100 kV and 10 mA to obtain a final irradiation of 0, 1, 2 and 4 Gray. Minutes after irradiation, cells have been counted and re-plated at the density of 200 cells/well in 6 wells plates and left to grow up to 14 days. Early cell death in response to treatment was measured 24 hours post-treatment using a propidium iodide dye-exclusion assay, which revealed that in the presence of CNC-AuNPs the cytotoxic properties of x-ray radiotherapy were significantly increased over control groups treated only with radiotherapy without pre-incubation with CNC-AuNPs (FIG. 19A). No acute toxicity was induced by the CNC-AuNPs alone, thus confirming the good biocompatibility of the proposed nanosystem along with its ability to act as a radiosensitizer.

### Colony Forming Assay on Glioblastoma Cells (FIG. 19B)

U251 cells have been plated at 100 k/plate in 6-well plates, left to attach overnight. At t0 cells have been treated with CNC*-Au-Glc functionalized with glucose at 10 g/ml or left untreated for 24 hrs, in a final volume of 2.0 ml. After CNC*-Au-Tm incubation cells have been Xray irradiated with Faxitron CP-160 (Faxitron Xray corp) at 100 kV and 10 mA to obtain a final irradiation of 0, 1, 2 and 4 Gray. Minutes after irradiation, cells have been counted and re-plated at the density of 200 cells/well in 6-well plates and left to grow up to 14 days. After, colonies have been stained with crystal violet and counted, considering valid colonies with more than 50 cells. Surviving fraction has been calculated as the ratio between counted colonies and plated cells. Results in FIG. 19B showed that CNC-AuNPs improved the capability of radiotherapy in limiting tumor cell proliferation.

1. A multifunctional hybrid CNC-Au-based nanoplatform containing at least one and no more than two orthogonal anchoring points.

2. The multifunctional hybrid CNC-Au-based nanoplatform according to claim 1, wherein at least one bioactive headgroup is conjugated to at least one orthogonal anchoring point.

3. The multifunctional hybrid CNC-Au-based nanoplatform according to claim 2, wherein said at least one bioactive headgroup is a carbohydrate.

4. The multifunctional hybrid CNC-Au-based nanoplatform according to claim 1, wherein gold nanoparticles (AuNPs) are embedded onto a cellulose nanocrystal matrix and have dimensions≤5 nm.

5. A conjugate containing the multifunctional hybrid CNC-Au-based nanoplatform according to claim 1.

6. A method for the treatment of tumors comprising administering the multifunctional hybrid CNC-Au-based nanoplatform according to claim 1 or a conjugate containing said multifunctional hybrid CNC-Au-based nanoplatform.

7. The method for treating tumors according to claim 6 wherein said tumors are selected from the group consisting of breast cancers, uterine cancers, brain and central nervous system cancers, lung cancers, liver cancers, kidney cancers, testicular cancers, pancreatic cancers, skin cancers, thyroid cancers, intestinal cancers, head and neck cancers, ovarian cancers, stomach cancers, colon cancers, bladder cancers, prostate cancers, urinary tract cancers, eye cancers, oral and oropharyngeal cancers, HIV/AIDS-related cancers, hematopoietic and lymphoid cancers, and sarcomas of various tissues.

8. A method of radiotherapy, immunotherapy, photodynamic therapy, or boron neutron capture therapy (BNCT) in cancer treatment diagnosis comprising administering the multifunctional hybrid CNC-Au-based nanoplatform according to claim 1 or a conjugate containing said multifunctional hybrid CNC-Au-based nanoplatform.

9. A process for obtaining a multifunctional hybrid CNC-Au-based nanoplatform comprising:
a) preparing CNC*by treating pristine cellulose nanocrystals with a sulfatation agent and a solvent to give cellulose nanocrystals with a controlled sulfation; and
b) preparing CNC*-Au-LA by treating the CNC* with a controlled sulfation obtained at step a) with lipoamide (LA), the HAuCl₄/NaBH₄ in a solution of methanol/water to obtain the hybrid nanoplatform of CNC*-Au-LA.

10. The process according to claim 2, wherein step (b) is a one pot reaction.

11. The process according to claim 9, wherein the sulfatation agent is a mixture of SO₃/pyridine and the solvent is dimethylsulfoxide (DMSO).

12. The process according to claim 9, further comprising purifying the CNC*-Au-LA by dialysis.

13. The multifunctional hybrid CNC-Au-based nanoplatform according to claim 1, wherein at least one orthogonal anchoring point is represented by an OH group of glucose residues of CNC and alkyne on an Au surface.

14. The multifunctional hybrid CNC-Au-based nanoplatform according to claim 3, wherein said carbohydrate is a sugar.

15. The multifunctional hybrid CNC-Au-based nanoplatform according to claim 3, wherein said carbohydrate is selected from the group consisting of glucose, mannose, lactose, fucose, rhamnose, and Lewis-type antigens.

16. The multifunctional hybrid CNC-Au-based nanoplatform according claim 4, wherein the gold nanoparticles (AuNPs) have dimensions of about 5.0±1.0 nm.

17. The conjugate containing the multifunctional hybrid CNC-Au-based nanoplatform according to claim 5, wherein the conjugate is a monofunctional or bifunctional conjugate.

18. The method for treating tumors according to claim 6, wherein said tumors are selected from the group consisting of triple negative breast cancer, glioblastoma and melanoma.

19. A method for the treatment of tumors according to claim 6, wherein the tumors are solid tumors.
